(19) United States
(12) Patent Application Publication
(10) Pub. No.: US 2005/0065082 A1
(43) Pub. Date: Mar. 24, 2005

(54) C-jun phosphorylation inhibitors

(76) Inventors: Hirofumi Doi, Chiba-shi (JP); Naoya Wada, Tokyo (JP); Hiroto Nakajima, Tokyo (JP)

Correspondence Address: KILYK & BOWERSOX, P.L.L.C., 53 A EAST LEE STREET, WARRENTON, VA, 20186, US

(21) Appl. No.: 10/509,307

(22) PCT Filed: Mar. 31, 2003
(86) PCT No.: PCT/JP03/04120

(30) Foreign Application Priority Data
Mar. 29, 2002 (JP) 2002-095291
Mar. 29, 2002 (JP) 2002-095390
Mar. 29, 2002 (JP) 2002-095442
Mar. 29, 2002 (JP) 2002-095486

(51) Int. Cl. A61K 38/52; C12N 9/99

(52) U.S. Cl. 514/12; 435/184

(57) ABSTRACT

In the present invention, BMAL1, BPL1, KIAA1491 complete (SEQ ID NO: 1), and KIAA0596CT (SEQ ID NO:2), have a function for interacting with JNK3, wherein these peptides, as well as peptides derived therefrom can be used to inhibit c-Jun from being phosphorylated and exhibiting its functions (such as transcription activation activity). Inhibition of the phosphorylation of c-Jun suppresses apoptosis, such as nerve cell apoptosis. Thus, the present invention also provides a drug, a pharmaceutical composition and a method for use in preventing and/or treating a neurodegenerative disease (such as polyglutamine disease and Alzheimer's disease).

### TECHNICAL FIELD

The present invention relates to an agent for inhibiting the phosphorylation of c-Jun, wherein the agent comprises a peptide that has a function for interacting with c-Jun N-terminal kinase 3 (hereinafter also referred to as “JNK3”), and a method for inhibiting the phosphorylation of c-Jun, wherein the method comprises using the peptide. The present invention also relates to an agent for inhibiting the ability of c-Jun to activate transcription, wherein the agent comprises the peptide, and a method for inhibiting the ability of c-Jun to activate transcription, wherein the method comprises using the peptide. The present invention further relates to the peptide, a polynucleotide encoding the peptide, a recombinant vector comprising the polynucleotide, a transformant transfected with the recombinant vector, and an antibody to the peptide. The present invention also relates to a method of producing the peptide, wherein the method comprises using the transformant, a method of identifying a compound that promotes or inhibits the interaction of the peptide with JNK3, and a method of identifying a compound that mediates or inhibits the expression of a polynucleotide encoding the peptide. The present invention also relates to a pharmaceutical composition containing the peptide, the polynucleotide, the vector, the transformant, the antibody, the agent for inhibiting the phosphorylation of c-Jun, or the inhibitor of the ability of c-Jun to activate transcription, and a method for preventing and/or treating disease(s) caused by the phosphorylation of c-Jun comprising using these.

### BACKGROUND OF THE INVENTION

c-Jun N-terminal kinase (hereafter also referred to as “JNK”) is a protein phosphoenzyme belonging to the MAP kinase superfamily. A MAP kinase is an enzyme that plays an important role in an intracellular signaling pathway for regulating cellular proliferation and differentiation, and is activated, for example, by proliferative stimulus. However, unlike a classical MAP kinase (ERK), JNK is not substantially activated. Rather, JNK is activated by a cellular stress such as DNA damage, ultraviolet rays, high osmotic pressure, ER stress, and active oxygen, as well as by an inflammatory cytokine such as a tumor necrosis factor or interleukin 1.

It has been known that JNK is activated in response to stress to play a role in apoptosis because JNK activation is observed in the cell death of the nerve-derived cell line PC12 from nerve growth factor removal. Recently, it has been reported that JNK controls caspase dependent apoptosis and causes caspase independent apoptosis. Furthermore, it has been revealed that many cell deaths that occur pathologically (as in neurodegenerative disease) in mammals are independent of caspase (non-Patent Reference).

Apoptosis is classified into transcription dependent cell death (for example, sympathetic cell death by removal of nerve nutritive factors and death by DNA damage) and transcription independent apoptosis (for example, death from exposure to ultraviolet rays and death induced by Fas). It has been suggested that JNK plays a role in both forms of apoptosis. For transcription dependent apoptosis, it is recognized that JNK phosphorylates the serines (S) at the 63 and 73 positions in the amino acid sequence of c-Jun to activate the c-Jun. For example, death of neurocytes from kainic acid administration is inhibited in a knockin mouse that is a mutant (A63, A73) produced by substituting the phosphorylation sites of c-Jun with alanines (non Patent Reference 2). c-Jun is a transactivator that has an activity for transcription activation, that is, an activity to bind to a site for regulating gene expression to promote gene transcription. Therefore, c-Jun is presumed to induce the transcription of apoptosis promoting factors such as Bim and FasL in its downstream.

Three kinds of JNK genes, that is, the JNK 1 gene, the JNK 2 gene, and the JNK 3 gene are known to be found in mammals. It is known that the JNK 3 gene is expressed specifically in cranial nerves, for example. The JNK3 gene is known to be expressed at a stage of shock such as an anoxic condition to bring about damage to cerebral functions. It is also reported that the excitatory death of neurocytes from kainic acid administration is inhibited by a JNK 3 gene knockout (non-Patent Reference 3).

The references cited hereinafter are listed below.
Patent document 1: International Publication No. WO 01/67299.
Non-Patent Reference 1: Kitanaka, C. et al., Molecular medicine (2000)37: 408-418.
Non-Patent Reference 2: Behrens, A. et al., Nature Genetics (1999)21: 326-329.
Non-Patent Reference 3: Yang, D. D. et al., Nature (1 997) 389:865-870.
Non-Patent Reference 4: Koyano, S. et al., FEBS Letters (1999) 457:385-388.
Non-Patent Reference 5: Gekakis N. et al., Science (1998) 280:1564-1569.
Non-Patent Reference 6: Bunger M. K. et al., Cell (2000) 103:1009-1017.
Non-Patent Reference 7: Nippon Rinsho (1996) 54(1):259-267.
Non-Patent Reference 8: Sambruck et al., “Molecular Cloning, A laboratory manual, 2^{nd} version” Cold Spring Harbor Laboratory, 1989.
Non-Patent Reference 9: Muramatsu Masami ed., “Labomanual Gene Technology” Maruzen K K., 1988.
Non-Patent Reference 10: Ehrlich H. E, ed. “PCR technology, Principle and Application of DNA amplification” Stockton Press, 1989.
Non-Patent Reference 11: Ulmer, Science (1983)219:666-.
Non-Patent Reference 12: Ikeda, M. et al., Biochemical and Biophysical Research Communications (1997) 233: 258-264.
Non-Patent Reference 13: Suzuki et al., Nature Genetics (1994) 8(2): 122-128.
Non-Patent Reference 14: Yasuda, S. et al., Genes to Cells (1999) 4:734-756.
Non-Patent Reference 15: Ura Seiji, et al., Jikken Igaku (2001) 19 :1839-1844.
Non-Patent Reference 16: Nature (1957) 179:160-161.

### SUMMARY OF THE INVENTION

It is an object of the present invention to find a substance for inhibiting a signaling pathway in which JNK3 plays a role, and to provide a means for preventing and/or treating disease(s) caused by the abnormal signaling pathway including disease(s) caused by cell apoptosis, such as a neurodegenerative disease.

The present inventors conducted concentrated research to achieve the above-mentioned object and have found proteins BMAL1 and BPL1, as well as proteins comprised of amino acid sequences represented by SEQ ID NO:1, SEQ ID NO:2, and SEQ No:3 in the Sequence List, which interact with JNK3. They also have clarified that BMAL1 and the protein comprised of the amino acid sequence represented by SEQ ID NO:2 bind to JNK3. Furthermore, they have demonstrated that all the proteins inhibit the phosphorylation of c-Jun by JNK3.

Accordingly, an aspect of the present invention is an agent for inhibiting the phosphorylation of c-Jun wherein the agent comprises one or more peptides selected from the following peptide group as active ingredients:
(i) BMAL1,
(ii) BPL1,
(iii) a peptide comprised of the amino acid sequence represented by SEQ ID NO:1 in the Sequence List,
(iv) a peptide comprised of the amino acid sequence represented by SEQ ID NO:2 in the Sequence List,
(v) a peptide comprised of the amino acid sequence represented by SEQ ID NO:3 in the Sequence List,
(vi) a peptide comprising at least one peptide of the above (i) to (v), wherein the peptide has a function for inhibiting the phosphorylation of c-Jun by JNK3,
(vii) a peptide comprised of at least 5 consecutive amino acid residues found in an amino acid sequence of at least one peptide of (i) to (v), wherein the peptide has a function for inhibiting the phosphorylation of c-Jun by JNK3,
(viii) a peptide having mutations of one to several amino acid residues in an amino acid sequence of at least one peptide of (i) to (v), wherein the peptide has a function for inhibiting the phosphorylation of c-Jun by JNK3,
(ix) a peptide having a homology of 70% or more to at least one peptide of (i) to (v), wherein the peptide has a function for inhibiting the phosphorylation of c-Jun by JNK3,
(x) a peptide comprised of the amino acid sequence represented by SEQ ID NO:4 in the Sequence List, and
(xi) a peptide comprised of the amino acid sequence represented by SEQ ID NO:5 in the Sequence List.

Furthermore, an aspect of the present invention is an agent for inhibiting the phosphorylation of c-Jun, wherein the agent is a peptide group consisting of (i), (ii), (iii), (iv), (x), or (xi).

In addition, an aspect of the present invention is a method for inhibiting the phosphorylation of c-Jun, wherein the method comprises putting one or more peptides selected from the peptide group having a function for interacting with JNK3 in coexistence with JNK3.

Furthermore, an aspect of the present invention is a method for inhibiting the phosphorylation of c-Jun, wherein the peptide group consists of (i), (ii), (iii), (iv), (x), or (xi).

In addition, an aspect of the present invention is an agent for inhibiting the ability of c-Jun to activate transcription, wherein the agent comprises one or more peptides selected from the peptide group having a function for interacting with JNK3 as active ingredients.

In addition, an aspect of the present invention is a method for inhibiting the ability of c-Jun to activate transcription, wherein the method comprises putting one or more peptides selected from the above peptide group having a function for interacting with JNK3 in coexistence with JNK3.

In addition, an aspect of the present invention is a pharmaceutical composition comprising an effective dose of the agent for inhibiting the phosphorylation of c-Jun or for inhibiting the ability of c-Jun to activate transcription.

In addition, an aspect of the present invention is a pharmaceutical composition, wherein the pharmaceutical composition is an agent for preventing and/or treating disease(s) caused by the phosphorylation of c-Jun by JNK3.

In addition, an aspect of the present invention is a pharmaceutical composition comprising an effective dose of one or more peptides selected from the above peptide group having a function for interacting with JNK3, wherein the pharmaceutical composition is an agent for preventing and/or treating disease(s) caused by the phosphorylation of c-Jun by JNK3.

In addition, an aspect of the present invention is a pharmaceutical composition comprising an effective dose of one or more polynucleotides encoding at least one peptide selected from the above peptide group having a function for interacting with JNK3, wherein the pharmaceutical composition is an agent for preventing and/or treating disease(s) caused by the phosphorylation of c-Jun by JNK3.

In addition, an aspect of the present invention is a pharmaceutical composition described above, wherein the disease caused by the phosphorylation of c-Jun by JNK3 is a neurodegenerative disease.

Furthermore, an aspect of the present invention is a pharmaceutical composition, wherein the neurodegenerative disease is polyglutamine disease, Huntington's disease, spinocerebellar ataxia, bulbospinal muscular atrophy, dentatorubropallidoluysian atrophy, Alzheimer's disease, Down's disease, Parkinson's disease, Lewy body dementia, multiple system atrophy, familial amyotrophic lateral sclerosis, progressive supranuclear palsy, corticobasal degeneration, Pick's disease, familial British dementia, Creutzfeldt-Jakob disease, Gerstmann-Stranssler syndrome, mad cow disease (bovine spongiform encephalopathy) (BSE), or familial dementia accompanying neuroserpin inclusion bodies.

In addition, an aspect of the present invention is a method for preventing and/or treating disease(s) caused by the phosphorylation of c-Jun by JNK3, wherein the method comprises putting one or more peptides selected from the peptide group having a function for interacting with JNK3 in coexistence with JNK3.

In addition, an aspect of the present invention is a method for preventing and/or treating disease(s) caused by the phosphorylation of c-Jun by JNK3, wherein the method comprises using one or more polynucleotides encoding at least one peptide selected from the peptide group having a function for interacting with JNK3 to inhibit the phosphorylation of c-Jun by JNK3 by expressing a peptide encoded by the polynucleotides.

In addition, an aspect of the present invention is a method for preventing and/or treating disease(s) caused by the phosphorylation of c-Jun by JNK3, wherein the method comprises using the pharmaceutical composition.

In addition, an aspect of the present invention is a method for preventing and/or treating disease(s), wherein the disease caused by the phosphorylation of c-Jun by JNK3 is a neurodegenerative disease.

Furthermore, an aspect of the present invention is a method for preventing and/or treating disease(s), wherein the neurodegenerative disease is polyglutamine disease, Huntington's disease, spinocerebellar ataxia, bulbospinal muscular atrophy, dentatorubropallidoluysian atrophy, Alzheimer's disease, Down's disease, Parkinson's disease, Lewy body dementia, multiple system atrophy, familial amyotrophic lateral sclerosis, progressive supranuclear palsy, corticobasal degeneration, Pick's disease, familial British dementia, Creutzfeldt-Jakob disease, Gerstmann-Stranssler syndrome, mad cow disease (Bovine Spongiform Encephalopathy) (BSE), or familial dementia accompanying neuroserpin inclusion bodies.

In addition, an aspect of the present invention is at least one peptide selected from the following peptide group:
(i) a peptide comprised of the amino acid sequence represented by SEQ ID NO:1 in the Sequence List,
(ii) a peptide comprising a peptide comprised of the amino acid sequence represented by SEQ ID NO:1 in the Sequence List,
(iii) a peptide comprised of at least 5 consecutive amino acid residues in the amino acid sequence represented by SEQ ID NO:1 in the Sequence List, and
(iv) a peptide having mutations of one to several amino acids found in at least one peptide of (i) to (iii).

Furthermore, an aspect of the present invention is the above peptide having a function for interacting with JNK3.

In addition, a further aspect of the present invention relates to a polynucleotide comprising a nucleotide sequence encoding the peptide or a complementary sequence thereof.

In addition, an aspect of the present invention relates to a polynucleotide comprised of a nucleotide represented by SEQ ID NO:7 in the Sequence List.

In addition, an aspect of the present invention is a polynucleotide that hybridizes with the polynucleotide under stringent conditions.

In addition, an aspect of the present invention is a recombinant vector comprising the polynucleotide.

Furthermore, an aspect of the present invention is a recombinant vector described above, wherein the recombinant vector is a recombinant expression vector.

In addition, an aspect of the present invention is a transformant transfected with the recombinant vector.

In addition, an aspect of the present invention relates to a method for producing the peptide, wherein the method comprises a process for culturing the transformant transfected with the recombinant vector.

Furthermore, an aspect of the present invention is an antibody that immunologically recognizes the peptide.

In addition, an aspect of the present invention is a method of identifying a compound that promotes or inhibits the interaction of the peptide with JNK3, comprising using at least one of the following: the peptide, a polynucleotide encoding the peptide, a recombinant vector comprising the polynucleotide, a transformant transfected with the recombinant vector, or an antibody that immunologically recognizes the peptide.

In addition, an aspect of the present invention is a method of identifying a compound that promotes or inhibits the expression of a polynucleotide encoding the peptide, wherein the method comprises using at least one of the following: a polynucleotide encoding the peptide, a recombinant vector comprising the polynucleotide, a transformant transfected with the recombinant vector, and an antibody that immunologically recognizes the peptide.

Furthermore, an aspect of the present invention is a pharmaceutical composition comprising an effective dose of at least one of the following: the peptide, a polynucleotide encoding the peptide, a recombinant vector comprising the polynucleotide, a transformant transfected with the recombinant vector, or an antibody that immunologically recognizes the peptide.

### BRIEF DESCRIPTION OF DRAWINGS

FIG. 1 illustrates the result of the in silico prediction of the interaction between JNK3 and KIAA1491 (SEQ ID NO:6). Local alignment between JNK3 and KIAA1491 was conducted, and regions with high scores are shown. The upper and lower rows indicate sequences present in JNK3 and sequences present in KIAA1491, respectively.

FIG. 2A and FIG. 2B show that c-Jun and KIAA1491 complete (SEQ ID NO: 1) were phosphorylated in vitro by JNK3. FIG. 2A shows that although GST-c-Jun (1-79) (a fusion protein of glutathione S-transferase (GST) and a 79-amino acid region at the N-terminus of c-Jun) was phosphorylated by JNK3, GST was not phosphorylated. FIG. 2B shows that a fusion protein of GST and KIAA1491 complete (GST-KIAA1491 complete) was dose-dependently phosphorylated by JNK3. The numerical values on the left indicate molecular weight.

FIG. 3 shows that KIAA1 491 complete (SEQ ID NO:1) inhibited the phosphorylation of c-Jun by JNK3. Lane 1 shows the result when another protein was not added to the reaction for the phosphorylation of c-Jun (1 -79) by JNK3, lanes 2 and 3 show the results when 1 μg and 2 μg of GST were added thereto, respectively, and lanes 4 and 5 show the results when 1 μg and 2 μg of GST-KIAA 1491 were added thereto, respectively. The arrowhead indicates phosphorylated c-Jun (1-79).

FIG. 4 illustrates the result of the in silico prediction of the interaction between JNK3 and KIAA0596 (SEQ ID NO: 3). Local alignment between JNK3 and KIAA0596 was conducted, and regions with high scores are shown. The upper and lower rows indicate sequences present in JNK3 and sequences present in KIAA0596, respectively.

FIG. 5 shows that KIAA0596CT (a peptide comprising the amino acid sequence from amino acid 639 to amino acid 1217 of the amino acid sequence of KIAA0596 (SEQ ID NO: 2)) bound with JNK3. The upper figure shows results detected the binding between GST-KIAA0596CT (fusion protein of KIAA0596CT and GST) and MBP-JNK3 (fusion protein of JNK3 and maltose binding protein (MBP)) by a far-Western method. The lower figure shows results detected the binding between GST-KIAA0596CT and MBP as a negative control by far-Western method.

FIG. 6 shows that KIAA0596CT (SEQ ID NO: 2) inhibited the phosphorylation of c-Jun by JNK3. Lanes 1 and 4 show the results when another protein was not added to the reaction for the phosphorylation of c-Jun (1-79) by JNK3, lanes 2 and 3 show the results when 1 μg and 2 μg of GST were added thereto, respectively, and lanes 5 and 6 show the results when 1 μg and 2 μg of GST-KIAA0596CT were added thereto, respectively. The arrowheads indicate phosphorylated c-Jun (1-79).

FIG. 7 illustrates the result of the in silico prediction of the interaction between JNK3 and BMAL1. Local alignment between JNK3 and BMAL1 was conducted, and regions with high scores are shown. The upper and lower rows indicate sequences present in JNK3 and sequences present in BMAL1, respectively.

FIG. 8 illustrates the result of the in silico prediction of the interaction between JNK3 and BMAL2. Local alignment between JNK3 and BMAL2 was conducted, and regions with high scores are shown. The upper and lower rows indicate sequences present in JNK3 and sequences present in BMAL2, respectively.

FIG. 9A and FIG. 9B show that BMAL1 was phosphorylated in vitro by JNK3. FIG. 9A shows the positions to which each of the purified GST fusion proteins (lane 1: GST-BMAL1, lane 2: GST, lane 3: GST-c-Jun (1-79)) migrated in sodium dodecyl sulfate-polyacrylamide gel electrophoresis (SDS-PAGE) (arrowheads) detected by Coomassie brilliant blue (CBB) staining. Lane M is a molecular weight marker. The numerical values on the left of the figure indicate molecular weight. FIG. 9B shows that although GST-BMAL1 (lane 1) and GST-c-Jun (1-79) (lane 3) were phosphorylated by JNK3, GST (lane 2) was not phosphorylated. The arrowheads indicate phosphorylated GST-BMAL1 and GST-c-Jun (1-79).

FIG. 10 shows that BMAL1 was dose-dependently phosphorylated by JNK3. Lane 1, lane 2, lane 3, and lane 4 show the results obtained using no JNK3 (BMAL1 alone), 14 ng of JNK3, 28 ng of JNK3, and 70 ng of JNK3, respectively. The arrowhead indicates phosphorylated GST-BMAL1. The numerical values on the left of the figure indicate molecular weight of molecular weight markers.

FIG. 11 shows that BMAL1 inhibited the phosphorylation of c-Jun by JNK3. Lanes 1 and 4 show the results obtained when another protein was not added to the reaction for the phosphorylation of c-Jun (1-79) by JNK3, lanes 2 and 3 show the results when 1 μg and 2 μg of GST were added thereto, respectively, and lanes 5 and 6 show the results when 1 μg and 2 μg of GST-BMAL1 were added thereto, respectively. The arrowheads indicate phosphorylated c-Jun (1-79).

FIG. 12 illustrates the result of the in silico prediction of the interaction between JNK3 and BPL1. Local alignment between JNK3 and BPL1 was conducted, and regions with high scores are shown. The upper and lower rows indicate sequences present in JNK3 and sequences present in BPL1, respectively.

FIG. 13 shows that BPL1 inhibited the phosphorylation of c-Jun by JNK3. Lane 1 shows the result obtained when another protein was not added to the reaction for the phosphorylation of c-Jun (1-79) by JNK3, lanes 2 and 3 show the results obtained when 1 μg and 2 μg of GST were added thereto, respectively, and lanes 4 and 5 show the results when 1 μg and 2 μg of GST-BPL1 were added thereto, respectively. The arrowhead indicates phosphorylated c-Jun (1-79).

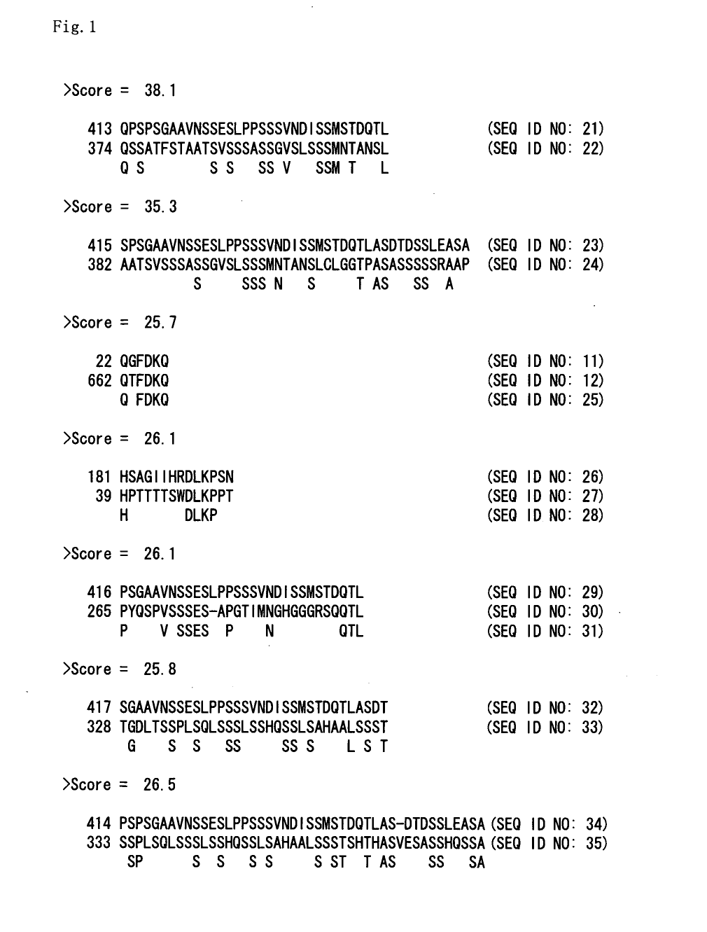

### DETAILED DESCRIPTION OF THE INVENTION

The present invention claims the benefit of priority from Japanese Patent Application Nos. 2002-95291, 2002-95390, 2002-95442 and 2002-95486, which are incorporated herein by reference.

Technical and scientific terms used herein have the meanings as normally understood by those skilled in the art, unless otherwise defined. Various methods that are well known to those skilled in the art are referenced herein. These reference materials, such as published materials disclosing known methods cited herein, are incorporated herein by reference in their entirety.

Embodiments of the present invention are explained in more detail below. However, the detailed description below is exemplary and only for the purpose of explanation, and is not intended to limit the scope of the present invention.

In the present invention, proteins that interact with JNK3 were predicted according to a method described in International Publication No. WO 01/67299 (Patent Document 1), and as a result the following proteins were found: BPL1, BMAL1, KIAA1491 (SEQ ID NO:6) and KIAA0596 (SEQ ID NO:3). Further, a nucleotide sequence (SEQ ID NO:7) encoding the full length of KIAA1491 (hereunder, referred to as “KIAA1491 complete”) was obtained by predicting in silico a region on the N-terminal side of KIAA1491 that was considered to be deleted, whereby KIAA1491 complete (SEQ ID NO:1) was acquired using a genetic engineering technique. It was clarified by experimentation that a protein KIAA0596CT (SEQ ID NO:2) comprised of an amino acid sequence of the C-terminal region of KIAA0596 (SEQ ID NO:3) and BPL1 bind with JNK3. It was also found that each of the proteins BPL1, BMAL1, KIAA1491 complete (SEQ ID NO:1) and KIAA0596CT (SEQ ID NO:2) inhibit the phosphorylation of c-Jun by JNK3.

In the present specification, the term “peptide” is used as a generic term to refer to an isolated or synthetic full-length protein, an isolated or synthetic full-length polypeptide, or an isolated or synthetic full-length oligopeptide, and in this specification the minimum size of a protein, a polypeptide or an oligopeptide is two amino acids. Amino acids may be designated by one-letter codes or three-letter abbreviations.

Further, in the present specification, the term “peptide having a function of interacting with JNK3” refers to a peptide that specifically interacts with JNK3, and specifically refers to, for example, a peptide that specifically contacts or binds with JNK3 as one of its functions. More specifically, it refers to a peptide that inhibits the ability of JNK3 to phosphorylate as one of the functions thereof, and preferably to a peptide that exhibits a competitively inhibiting function in the coexistence of an effective dose of the peptide and JNK3.

It is reported that the phosphorylation of c-Jun by JNK3 is involved in apoptosis, for example, in the death of neurocytes (Non-patent Document 2). Further, c-Jun is a transactivator that has the ability to activate transcription whereby it binds to a gene expression regulatory site to promote transcription of the gene, and induce transcription of an apoptosis promoter such as, for example, Bim or FasL, downstream of the signaling pathway thereof.

Therefore, by inhibiting the phosphorylation of c-Jun by. JNK3 using the aforementioned peptides that interact with JNK3, the ability of c-Jun to activate transcription can be inhibited. Further, it is also possible to suppress physiological phenomenon caused by the phosphorylation of c-Jun by JNK3, such as apoptosis, and therefore to inhibit and/or treat disease(s) caused by the phosphorylation of c-Jun by JNK3, such as a neurodegenerative disease.

A peptide KIAA1491 (SEQ ID NO:6) that was predicted to interact with JNK3 is a peptide (GenBank accession number: AB040924) that was found in the HUGE database of Kazusa DNA Research Institute. The functions of this peptide had not been previously reported. Since it was considered that a region on the N-terminal side of KIAA1 491 was deleted, the deletion region was predicted in silico, and it was found that a cDNA sequence encoding the region corresponds to a sequence of QVI-MT0132-201100-498-b05 MTO132 Homo sapiens cDNA (GenBank accession number: BF894928). Thus, a nucleotide sequence (SEQ ID NO:7) encoding the full length of KIAA1491 (KIAA1491 complete) was obtained. A full-length open reading frame (ORF) of the KIAA1491 complete gene is comprised of 2361 base pairs, and a gene product of the gene is comprised of 786 amino acid residues (SEQ ID NO:1). There have been no reports to date concerning this gene or the gene product thereof. Further, it was also clarified that mRNA corresponding to KIAA1491 complete cDNA is present in the brain, as shown by a reverse transcription polymerase chain reaction (RT-PCR) using poly-A RNA derived from human brain.

KIAA1491 complete was obtained from cDNA of human brain by a genetic engineering technique. As the result of experimental studies concerning the interaction of KIAA1491 complete with JNK3, it was found that KIAA1491 complete is phosphorylated by JNK3, and also that KIAA1491 complete inhibits the phosphorylation of c-Jun by JNK3. That is, it is considered that KIAA1491 complete interacts with JNK3 to inhibit the phosphorylation of c-Jun by JNK3.

KIAA0596 (SEQ ID NO:3) (GenBank accession number: AB 01168) is another of the aforementioned peptides. There had been no report regarding the functions of KIAA0596. It was clarified that KIAA0596 has a homology of 74% to mouse JNK-binding protein 1 (JNKBP1) (GenBank accession number: AB029482)), which has been reported as binding with JNK3 (Non-patent Document 4). Thus, it was predicted that KIAA0596 is a human homolog of JNKBP1. There has been no report to date concerning JNKBP1 in humans. Mouse JNKBP1 is an activator protein of the JNK signaling pathway, which promotes the activity of JNK3 for the phosphorylation of c-Jun. There is a possibility that Mouse JNKBP1 plays a similar role to JIP-1, which is a scaffold protein in the JNK signaling pathway (Non-patent Document 4). Since it was known that the C-terminal region of mouse JNKBP1 is required for binding with JNK3, we prepared a peptide (SEQ ID NO:2, hereinafter referred to as “KIAA0596CT”) comprised of a C-terminal region (amino acid sequence from amino acid 639 to 1217) of KIAA0596. We found that KIAA0596CT binds with JNK3 and dose-dependently inhibits the phosphorylation of c-Jun by activated JNK3. More specifically, it is supposed that KIAA0596CT binds with JNK3 to inhibit the activity of JNK3 for the phosphorylation of c-Jun, to thereby inhibit the phosphorylation of c-Jun. In addition, it is supposed that KIAA0596 (SEQ ID NO:3), which comprises KIAA0596CT, binds to JNK3 in the same manner as KIAA0596CT to inhibit the phosphorylation of c-Jun by JNK3.

BMAL1, still another of the aforementioned peptides, interacted with JNK3 to inhibit the phosphorylation of c-Jun by JNK3, although it was likewise phosphorylated. Specifically, it is supposed that BMAL1 interacts with JNK3 to inhibit the activity of JNK3 for phosphorylation of c-Jun, to thereby inhibit the phosphorylation of c-Jun.

BMAL1 is a transcription factor that promotes transcription of the PERIOD gene (PER), a circadian rhythm-associated gene. BMAL1 also forms a heterodimer with the CLOCK protein to bind to an E-box sequence that is present in a PER gene promoter (Non-patent Document 5 and Non-patent Document 6). In knockout mice of MOP3, an isoform of BMAL1, a circadian rhythm abnormality is observed.

BPL1, still another of the aforementioned peptides, bound to JNK3 and dose-dependently inhibited the phosphorylation of c-Jun by JNK3. That is, it is supposed that BPL1 binds with JNK3 to inhibit the activity of JNK3 for the phosphorylation of c-Jun, to thereby inhibit the phosphorylation of c-Jun.

BPL1 is a holocarboxylase synthetase, and it has a function for binding biotin with carboxylase, in particular, four kinds of metabolism-related carboxylases: methylcrotonyl-CoA carboxylase, acetyl-CoA carboxylase, pyruvate carboxylase, and propionyl-CoA carboxylase to activate the carboxylase (Non-patent Document 7). A shortage of the enzyme causes holocarboxylase synthetase deficiency. Holocarboxylase synthetase deficiency is an autosomal recessive inborn error of metabolism indicated by a diversity of biochemical abnormalities and clinical symptoms, which reflect a decline in the activity of a plurality of carboxylases (Non-patent Document 7).

Based on the above findings, the present invention provides an agent for inhibiting the phosphorylation of c-Jun and an agent for inhibiting the ability of c-Jun to activate transcription comprising a peptide that interacts with JNK3 as an active ingredient. The present invention also provides a method for inhibiting the phosphorylation of c-Jun and a method for inhibiting the ability of c-Jun to activate transcription, in one respect, comprising putting peptides that interact with JNK3 in coexistence with JNK3. As used herein, the term “agent for inhibiting the ability of c-Jun to activate transcription” refers to a substance that acts on a mechanism of transcription activation that comprises c-Jun, in some manner including binding of c-Jun to a specific DNA sequence or binding to another factor that conducts transcription in cooperation with c-Jun, ultimately to inhibit transcriptional activity that involves c-Jun.

Preferably, a peptide that interacts with JNK3 is KIAA1491 complete (SEQ ID NO: 1), KIAA0596CT (SEQ ID NO: 2), KIAA0596 (SEQ ID NO: 3), BMAL1 or BPL1. Although BMAL1 and BPL1 are known peptides, it has not been reported that they inhibit the phosphorylation of c-Jun by JNK3. Several isoforms of BMAL1 are known, for example, BMAL1a and BMAL1b, and they can each be used in the present invention. Preferably, BMAL1b can be used. Peptides that have a function for inhibiting the phosphorylation of c-Jun and are derived from any of these peptides are also included in the scope of the present invention. A peptide derived from KIAA1491 complete (SEQ ID NO: 1), KIAA0596CT (SEQ ID NO: 2), KIAA0596 (SEQ ID NO: 3), BMAL1 or BPL1 is, for example, a peptide comprising any of these five peptides, or a partial peptide of any of these five peptides. Alternatively, the peptide may be a peptide having a mutation in the amino acid sequence thereof relative to an amino acid sequence of any of these five peptides, or a peptide having a homology of 70% or more to any of these five peptides. The agent for inhibiting the phosphorylation of c-Jun and the agent for inhibiting the ability of c-Jun to activate transcription according to the present invention comprise one or more peptides selected from the above peptides as active ingredients. Further, the method for inhibiting the phosphorylation of c-Jun and the method for inhibiting the ability of c-Jun to activate transcription according to the present invention comprise, in one respect, putting one or more peptides selected from the above peptides in coexistence with JNK3. Coexistence of the peptide(s) with JNK3 can be carried out in vitro or in vivo.

As used herein, the term “partial peptide” refers to a peptide comprised of at least 5, preferably 8 or more, more preferably 12 or more, and even more preferably 15 or more consecutive amino acid residues in the amino acid sequence of the peptide comprising the partial peptide. More preferably, a peptide that comprises a specifically acting site or binding site, such as a contact site between JNK3 and a peptide that interacts with JNK3, is desirable. Since it is supposed that the partial peptide can, for example, competitively inhibit the phosphorylation of c-Jun by JNK3, the partial peptide is suitable for inhibiting the phosphorylation of c-Jun by JNK3.

The term “a peptide having mutation in the amino acid sequence thereof relative to an amino acid sequence of any of the above peptides” refers to a peptide comprised of an amino acid sequence having mutation such as a deletion, substitution, addition or insertion of one or more amino acids, for example, 1 to 100, preferably 1 to 30, more preferably 1 to 20, even more preferably 1 to 10, and most preferably I to several amino acids in the amino acid sequence in question. A peptide having the mutation may be a natural peptide or a peptide in which a mutation is introduced. Methods for introducing mutations are well known. For example, methods involving site-specific mutagenesis, genetic homologous recombination, primer extension, polymerase chain reaction (PCR) or the like can be used alone or in suitable combinations thereof. The above methods can be carried out, for example, according to methods described in publications (Non-patent Document 8, Non-patent Document 9 and Non-patent Document 10), or using a modified method thereof. For example, the Ulmer's technique (Non-patent Document 11) may be utilized. In the introduction of the mutation, in order to avoid a change in the fundamental properties (physical properties, function, physiological activity, immunological activity, and the like) of the peptide, for example, mutual substitution among related amino acids (polar amino acids, non-polar amino acids, hydrophobic amino acids, hydrophilic amino acids, positive-charged amino acids, negative-charged amino acids and aromatic amino acids or the like) may be considered. In addition, with respect to these available peptides, an amino group or carboxyl group constituting the peptides can be altered, for example, by amidation or the like, to the extent that doing so does not cause a remarkable change in their function.

KIAA1491 complete (SEQ ID NO: 1), KIAA0596CT (SEQ ID NO: 2), KIAA0596 (SEQ ID NO: 3), BMAL1 and BPL1 can be obtained by common genetic engineering techniques. For example, using suitable primers designed by known methods based on the information of polynucleotides encoding the peptides, it is possible to amplify a gene of interest using a human-brain derived cDNA library as a template. Thereafter, it is possible to obtain cells that express the peptides by inserting the obtained gene into an expression vector, and then introducing the vector into a host cell. As a further example, it is also possible to synthesize the peptides utilizing a known cell-free protein synthesis system.

As a polynucleotide encoding KIAA1491 complete, for example, the polynucleotide described in SEQ ID NO: 7 can be used. As a polynucleotide encoding KIAA0596CT (SEQ ID NO: 2) and KIAA0596 (SEQ ID NO: 3), for example, the polynucleotide described in SEQ ID NO: 8 can be used. BMAL1 is a known peptide (Non-patent Document 12) and a polynucleotide encoding BMAL1 has been disclosed. It is possible to acquire BMAL1 by utilizing the sequence information thereof. For example, a polynucleotide encoding a peptide comprised of an amino acid sequence described in SEQ ID NO:4, such as a polynucleotide comprised of the nucleotide sequence described in SEQ ID NO:9, can be used. The cDNA of human BPL1 has already been cloned (Non-patent Document 13), and an amino acid sequence thereof has been disclosed. For example, BPL1 can be obtained by a known genetic engineering technique using a polynucleotide encoding the peptide comprised of the amino acid sequence described in SEQ ID NO: 5, such as a polynucleotide comprised of the nucleotide sequence described in SEQ ID NO: 10.

Peptides derived from KIAA1491 complete (SEQ ID NO: 1), KIAA0596CT (SEQ ID NO: 2), KIAA0596 (SEQ ID NO: 3), BMAL1 or BPL1 can be obtained by designing the peptides on the basis of the respective amino acid sequences and performing synthesis according to known methods. For example, solid phase synthesis and liquid phase synthesis and the like are known methods for the chemical synthesis of a peptide, and any of these methods may be used. Further, peptides may also be obtained by common methods using genetic engineering techniques. Alternatively, partial peptides of the above peptides can be obtained by cleaving the above peptides using a suitable peptidase. Preferably, a peptide that interacts with JNK3 may be further selected from the obtained peptides. For example, for peptides derived from KIAA0596CT (SEQ ID NO: 2), KIAA0596 (SEQ ID NO: 3) or BPL1, a peptide having a function for binding with JNK3 may be selected by employing binding with JNK3 as an indicator. Alternatively, a peptide that interacts with JNK3 to inhibit the phosphorylation of c-Jun may be selected. Inhibition of the phosphorylation of c-Jun by a peptide can be assessed by, for example, putting a peptide derived from KIAA1491 complete (SEQ ID NO: 1), KIAA0596CT (SEQ ID NO: 2), KIAA0596 (SEQ ID NO: 3), BMAL1 or BPL1 in coexistence with JNK3 under the conditions where c-Jun is phosphorylated by JNK3, and determining whether the phosphorylation is inhibited or not. Detection of phosphorylation of c-Jun can be carried out by a common method for detecting a phosphorylated protein.

KIAA1491 complete (SEQ ID NO: 1), KIAA0596CT (SEQ ID NO: 2), KIAA0596 (SEQ ID NO: 3), BMAL1 and BPL1, or a peptide derived from any of them, have a function for interacting with JNK3 and exhibit an inhibitory action for the phosphorylation of c-Jun by JNK3.

In recent years, it has been reported that the JNK signaling pathway is activated in a polyglutamine related aggregation in a model system of cell death caused by a polyglutamine disease. It has also been reported that MKK4 (MAP kinase kinase 4, which is located upstream of JNK in JNK signaling and phosphorylates JNK to activate) or a dominant-negative mutant of c-Jun inhibits cell death caused by polyglutamine (Non-patent Document 14). Data has also been disclosed that indicates that JNK is involved in cell death caused by amyloid β, which is a cleavage product of amyloid precursor protein (APP), a causative gene of Alzheimer's disease (Non-patent Document 15).

It is therefore believed that by inhibiting the phosphorylation of c-Jun by JNK3 using KIAA1491 complete (SEQ ID NO: 1), KIAA0596CT (SEQ ID NO: 2), KIAA0596 (SEQ ID NO: 3), BMAL1 or BPL 1, or a peptide derived from any of these, it is possible to resolve as well as inhibit and/or treat disease(s) caused by apoptosis involving the phosphorylation of c-Jun by JNK3 such as, for example, a neurodegenerative disease.

Examples of neurodegenerative disease include, but are not limited to, polyglutamine disease (for example, Huntington's disease, spinocerebellar ataxia, bulbospinal muscular atrophy, and dentaterubropallidoluysian atrophy), Alzheimer's disease, Down's disease, Parkinson's disease, Lewy bodies dementia, multiple system atrophy, familial ALS (amyotrophic lateral sclerosis), progressive supranuclear palsy, corticobasal degeneration, Pick's disease, familial British dementia, Creutzfeldt-Jakob disease, Gerstmann-Stranssler syndrome, mad cow disease (bovine spongiform encephalopathy) (BSE), and familial dementia accompanying neuroserpin inclusion bodies.

By utilizing KIAA1491 complete (SEQ ID NO: 1), KIAA0596CT (SEQ ID NO: 2), KIAA0596 (SEQ ID NO: 3), BMAL1 or BPL1 or a peptide derived from any of them, independently or in a combination of a plurality thereof, the present invention provides a pharmaceutical composition, which comprises at least one of the peptides as an active ingredient. A pharmaceutical composition comprising an effective dose of the agent for inhibiting the phosphorylation of c-Jun or the agent for inhibiting the ability of c-Jun to activate transcription according to the present invention is also included within the scope of the present invention. The pharmaceutical composition according to the present invention can be used as a preventing and/or treating agent for disease(s) caused by the phosphorylation of c-Jun by JNK3, such as a neurodegenerative disease.

It is also possible to inhibit and/or treat the aforementioned diseases by expressing in vivo a polynucleotide encoding any member of KIAA1491 complete (SEQ ID NO: 1), KIAA0596CT (SEQ ID NO: 2), KIAA0596 (SEQ ID NO: 3), BMAL1 or BPL1 or a peptide derived from any of them using a vector for protein expression or a transporter, to inhibit the phosphorylation of c-Jun by JNK3. Expression of these peptides in vivo can be carried out utilizing known methods. For example, in vivo expression can be performed by obtaining a polynucleotide encoding one of the peptides, then inserting it into a nucleic acid vector such as a replication-defective retrovirus vector, and delivering it into a target cell. Accordingly, a pharmaceutical composition comprising at least one of the polynucleotides encoding at least one of KIAA1491 complete (SEQ ID NO: 1), KIAA0596CT (SEQ ID NO: 2), KIAA0596 (SEQ ID NO: 3), BMAL1 or BPL1 or a peptide derived from any of them as an active ingredient, that is, the agent for preventing and/or treating the disease caused by the phosphorylation of c-Jun by JNK3, is also included within the scope of this invention.

The present invention also provides a method for preventing and/or treating the disease caused by the phosphorylation of c-Jun by JNK3, which comprises putting KIAA1491 complete (SEQ ID NO: 1), KIAA0596CT (SEQ ID NO: 2), KIAA0596 (SEQ ID NO: 3), BMAL1 or BPL1 or a peptide derived from any of them in coexistence with JNK3. Further, the method for preventing and/or treating the disease caused by the phosphorylation of c-Jun by JNK3 that uses the agent for inhibiting the phosphorylation of c-Jun or the agent for inhibiting the ability of c-Jun to activate transcription according to this invention is also included within the scope of this invention. In addition, the present invention also provides a method for preventing and/or treating disease(s) caused by the phosphorylation of c-Jun by JNK3 which comprises using at least one polynucleotide encoding at least one of KIAA1491 complete (SEQ ID NO: 1), KIAA0596CT (SEQ ID NO: 2), KIAA0596 (SEQ ID NO: 3), BMAL1 or BPL1 or the peptide derived from any of them and expressing the peptide encoded by the polynucleotide to inhibit the phosphorylation of c-Jun by JNK3.

A formulation of the pharmaceutical composition of the present invention preferably includes a suitable pharmaceutical carrier. The formulation comprises a therapeutically effective dose of at least one of the group consisting of KIAA1491 complete (SEQ ID NO: 1), KIAA0596CT (SEQ ID NO: 2), KIAA0596 (SEQ ID NO: 3), BMAL1, BPL1, peptides derived from these peptides, polynucleotides encoding the peptides, and vectors comprising the polynucleotides, and a pharmaceutically acceptable carrier or excipient. Examples of the carrier include, but are not limited to, a physiological saline, buffered physiological saline, dextrose, water, glycerol, ethanol, and a mixture thereof. The formulation may be selected in accordance with the suitability thereof to an administration route, and the formulations are well known to those skilled in the art. At the time of formulation, it may be used alone or in combination with other compounds or medicine necessary for treatment.

The formulation may be for systemic administration or for local administration. A preferred mode of systemic administration is injection, such as, for example, intravenous injection. Other injection routes, such as subcutaneous, intramuscular and intraperitoneal injection, can also be used. Another mode of administration can be peroral administration, if an enteric-coated formulation or capsule formulation is suitably formulated. In addition, permucosal administration or percutaneous administration using a penetrating agent such as bile acid salt, fusidic acid, or other surfactants can also be used. Topical administration can be in the form of a plaster, paste, gel, etc.

The range of dosage required can be determined according to the effectiveness of at least one selected from the aforementioned peptides, polynucleotides, and vectors in a therapeutically effective dose; the administration route; the properties of the formulation; the nature of the symptoms to be treated; and the judgment of the doctor in attendance. Specifically, an adequate dose may, for example, be in the range of 0.1 μg to 100 μg per 1 kg of body weight of subject. However, the dose can be modified by means of common conventional experiments for optimization, which means are well known in the art.

In the terms of pharmaceutical preparation, well-known methods of pharmaceutical preparation may be introduced according to the properties of the various targets such as peptides, polynucleotides, oligonucleotides or the like. Specifically, known methods for preparing pharmaceutical compositions such as powdered drugs, pills, tablets, capsules, aqueous solutions, ethanol solutions, liposome preparations, fat emulsions, or clathrates(such as those of cyclodextrin) can be used.

Powder drugs, pills, capsules and tablets can be prepared using excipients such as lactose, glucose, sucrose or mannitol; disintegrants such as starch or sodium alginate; lubricants such as magnesium stearate and talc; binders such as polyvinyl alcohol, hydroxypropyl cellulose and gelatin; surfactants such as fatty acid esters; and plasticizers such as glycerin. For the preparation of a tablet or a capsule, a solid pharmaceutical carrier is used.

A suspension can be produced using water, sugars such as sucrose, sorbitol and fructose, glycols such as polyethylene glycol (PEG), and oils.

Injectable solutions can be prepared using a saline solution, a glucose solution or a carrier comprising a mixture of saline water and glucose solution.

Inclusion into liposomes can be performed, for instance, by adding a solution that is prepared by dissolving the substance of interest in a solvent (such as ethanol) to a solution that is prepared by dissolving phospholipid in an organic solvent (such as chloroform), then removing the solvent by evaporation, then adding a phosphate-buffered solution thereto, followed by agitating and sonicating thereof, and finally, centrifuging thereof to obtain the supernatant and filtering it for recovery.

Fat emulsification can, for example, be prepared by mixing the substance of interest, oil ingredients (vegetable oils such as bean oil, sesame oil and olive oil as well as MCT and the like), emulsifying agents (such as phospholipids) and the like, followed by heating to obtain a solution, then adding a necessary amount of water, and emulsifying/homogenizing with homogenizer (for example, high-pressure-spray type, sonicating type or the like). The thus obtained product may also be subjected to freezing and drying. Moreover, when emulsifying fat, an auxiliary emulsifier may be added. Examples of the auxiliary emulsifier include glycerin and sugars (such as glucose, sorbitol, or fructose).

Cyclodextrin clathrates can, for example, be prepared by adding a solution that is prepared by dissolving cyclodextrin in water or the like by heating to a solution that is prepared by dissolving the substance of interest in a solvent (such as ethanol), then cooling and filtering the precipitate, and dry-sterilizing. At this point, the cyclodextrin to be used can be suitably selected from cyclodextrins with different void diameters (α, β, or γ type) according to the size of the substance.

The present invention further provides the KIAA1491 complete peptide that is comprised of the amino acid sequence described in SEQ ID NO: 1 in Sequence List. The scope of the present invention also includes a peptide that is derived from KIAA1491 complete, such as a peptide comprising the amino acid sequence described in SEQ ID NO: 1 or a peptide (partial peptide) comprised of at least 5 consecutive amino acid residues in the amino acid sequence. Based on the amino acid sequence of KIAA1491 complete or a peptide derived from KIAA1491 complete, the present invention also provides a peptide comprised of an amino acid sequence having mutation such as a deletion, substitution, addition or insertion of one or more amino acids, for example, 1 to 100, preferably 1 to 30, more preferably 1 to 20, more preferably 1 to 10, and most preferably 1 or several amino acids, by employing interaction with JNK3, for example, the phosphorylation by JNK3 or inhibition of the phosphorylation of c-Jun by JNK3, as an indicator. A peptide having the mutation may be a natural peptide or a peptide in which a mutation is introduced. Introduction of a mutation such as a deletion, substitution, addition or insertion can be carried out according to the methods described above.

KIAA1491 complete or a peptide derived therefrom may be prepared from a cell in which they are expressed by a genetic engineering technique, or may be obtained as the synthesized product of a cell-free system, a chemically synthesized product, or a substance prepared from a cell or a sample from a living organism, as well as the further purified products of these. Further, as long as there is no influence on the property or function thereof, for example, interaction with JNK3, such as inhibition of the phosphorylation of c-Jun by JNK3 or the phosphorylation by JNK3, KIAA1491 complete or the peptide derived therefrom may be labeled. The labeling may be performed by adding another protein or peptide or the like at the N-terminal or C-terminal thereof directly, or indirectly through a linker peptide or the like using a genetic engineering technique. Preferably, the labeling is conducted in a manner that does not inhibit the fundamental properties of KIAA1491 complete or the peptide derived therefrom. Examples of a protein or peptide or the like that may be added include, but are not limited to, an enzyme such as glutathione S-transferase, β-galactosidase, horseradish peroxidase (HRP) or alkaline phosphatase; a tag peptide such as His-tag, Myc-tag, HA-tag, FLAG-tag, or Xpress-tag; a fluorescent substance such as green fluorescent protein, fluorescein isothiocyanate or phycoerythrin, a maltose binding protein, Fc fragment of immunoglobulin, biotin and a radioisotope. At the time of labeling, the proteins and peptides and the like may be added independently or a plurality thereof may be combined and added. By measuring the protein or peptide, or the like that is used in the above labeling, or a function thereof, for example, it is possible to detect interaction between the peptides and JNK3. Detection or purification of the peptide of the present invention is also facilitated.

Of peptides derived from KIAA1491 complete, a peptide that interacts with JNK3 is useful as a substance that inhibits the phosphorylation of c-Jun by JNK3 and is also useful for identifying a compound that regulates interaction between JNK3 and KIAA1491 complete. A peptide that is immunologically recognizable, such as, for example, an epitope peptide, can be used as an antigen to produce an antibody for KIAA1491 complete, either by itself or by binding to a carrier (for example, keyhole limpet hemocyanin or egg albumin), as described hereunder.

The present invention provides a polynucleotide comprising the nucleotide sequence encoding KIAA1491 complete or the peptide derived therefrom, or comprising the complementary sequence thereof. For example, the polynucleotide may be the polynucleotide comprising the nucleotide sequence encoding the peptide comprised of the amino acid sequence described in SEQ ID NO:1 in the Sequence List, or the complementary sequence thereof. Preferably, the polynucleotide is comprised of a nucleotide sequence described in SEQ ID NO:7 in the Sequence List. The polynucleotides provide useful genetic information, for example, for producing KIAA1491 complete or a peptide derived therefrom. And the polynucleotides can also be used as standard preparations or reagents for the nucleic acids.

The present invention further provides a polynucleotide hybridizing under stringent conditions to a corresponding region of the polynucleotide comprising the nucleotide sequence encoding the amino acid sequence of KIAA1491 complete or the peptide derived therefrom, for example, the amino acid sequence described in SEQ ID NO:1 in the Sequence List, or the complementary sequence thereof. The hybridization conditions can be in accordance with, for example, a method described in publications (Non-patent Documents) or the like. The polynucleotide need not necessarily comprise the complementary sequence of a polynucleotide of interest, as long as it hybridizes to the polynucleotide of interest, such as a polynucleotide comprised of the nucleotide sequence described in SEQ ID NO:7 in the Sequence List or the complementary strand thereof. For example, the polynucleotide is preferably a polynucleotide that exhibits a homology of 85% or more to a nucleotide sequence of the gene encoding KIAA1491 complete or the complementary sequence thereof, more preferably 90% or more, and even more preferably 95% or more. The present invention also includes a polynucleotide or oligonucleotide comprised of 10 or more consecutive nucleotides located in the designated region of the nucleotide sequence of the polynucleotides, preferably 15 or more consecutive nucleotides, and more preferably 20 or more consecutive nucleotides. The polynucleotide may be a polynucleotide that includes a gene added at the 5′-terminal or C′-terminal thereof, such as, for example, a gene for expressing enzymes such as glutathione S-transferase, β-galactosidase, horseradish peroxidase (HRP) or alkaline phosphatase; tag-peptides such as His-tag, Myc-tag, HA-tag, FLAG-tag or Xpress-tag; and the green fluorescent protein, as long as the gene does not inhibit the function for expressing the encoded peptide or the function of an expressed peptide. The polynucleotides can be used for producing KIAA1491 complete or the peptide derived therefrom. They also can be used as probes or primers for detecting mRNA or the gene encoding KIAA1491 complete, or as antisense oligonucleotides or the like for controlling expression of the gene. In this respect, the polynucleotides include not only translation regions but also sequences corresponding to non-translation regions. In order to specifically inhibit the expression of the gene encoding KIAA1491 complete by the antisense oligonucleotide, it is preferable to use a nucleotide sequence of a characteristic region of the gene. The polynucleotide encoding KIAA1491 complete or the peptide derived therefrom can be obtained, for example, by verifying the expressed protein with a known protein expression system, using a physiological activity thereof as an indicator. For example, interaction with JNK3, specifically, inhibition of the phosphorylation of c-Jun by JNK3 or the phosphorylation by JNK3 can be used as an indicator. When a cell-free protein expression system is used as the expression system, for example, a technique using a ribosome system derived from embryo or rabbit reticulocyte or the like can be used (Non-patent Document 16).

Recombinant vectors can be obtained by incorporating the polynucleotides into appropriate vector DNA. The vector DNA may be appropriately selected according to the kind of host and purpose of use. The vector DNA may be a vector extracted from a natural source, or one in which a section of DNA other than a section required for proliferation is partially deleted. Examples of vector DNA include a vector derived from a chromosome, episome or virus, such as a vector derived from a bacteria plasmid, a bacteriophage, a transposon, a yeast episome, an inserted element or a yeast chromosome element, for example, a vector derived from a virus such as baculovirus, papovavirus, SV40, vaccinia virus, adenovirus, fowlpox virus, pseudo-rabies virus or retrovirus, as well as a combined vector thereof, or for example, a vector derived from a genetic element of a plasmid or bacteriophage, such as a cosmid or a phagemid. According to the purpose of use, an expression vector or a cloning vector or the like may be used.

The recombinant vector comprises the genetic sequence of interest and genetic sequences that contain information for duplication and control, such as a promoter, a ribosome-binding region, a terminator, a signal sequence and an enhancer. The recombinant vector is prepared by combining these by a known method. Recombining a polynucleotide into the vector DNA can be carried out in accordance with known methods. For example, a method may be used in which a suitable restriction enzyme is selected and treated to cleave DNA at a specific site, the cleaved DNA is subsequently mixed with vector DNA treated in the same manner to recombine using a ligase. Alternatively, a desired recombinant vector can be obtained by ligating a suitable linker to a polynucleotide of interest and inserting it into a multicloning site of a vector.

A transformant can be obtained by introducing a vector DNA into a known host by known methods. The vector DNA to be introduced into a host may be one single kind of vector DNA or a mixture of two or more kinds of vector DNA. Examples of a host include Escherichia coli, yeasts, grass bacillus, insect cells, and animal cells. Introduction of the vector DNA into a host cell may be carried out by applying well-known methods such as standard methods described in publications (Non-patent Document 8). Considering the stability of the gene, introducing vector DNA into a host cell by integration into the chromosome is a more preferred method. However, an autonomous replication system using an extranuclear gene can be used as a simpler method. Specific examples thereof include calcium phosphate transfection, DEAE-dextran mediating transfection, microinjection, positive lipid mediating transfection, electroporation, genetic transduction, scrape loading, ballistic introduction, and infection.

In the examples described hereinafter, an insect cell is used. However, the host is not limited thereto.

It is possible to obtain KIAA1491 complete or the peptide derived therefrom by a known genetic engineering technique based on information relating to the nucleotide sequence thereof, such as, for example, the nucleotide sequence described in SEQ ID NO:7 in the Sequence List. For example, an amplified gene of interest from a human brain-derived cDNA library may be obtained using a designed primer, according to the nucleotide sequence in question, that is synthesized by known methods. After inserting the amplified gene into an expression vector for introduction into a host cell to thereby construct a cell that expresses the peptide, the peptide of interest may be obtained from the cell by known methods for extracting and purifying. A known cell-free protein synthesis system can also be used for synthesizing. In the example described hereinafter, a gene expression system that uses an insect cell is used. KIAA1491 complete is obtained by producing a baculovirus having incorporated therein an ORF part of KIAA1491 complete that was amplified by PCR, and then infecting an insect cell, Sf9, with the baculovirus to induce expression. However, a method of acquiring KIAA1491 complete is not limited to the method described in this example.

For example, an expression vector may be used as vector DNA, and a polynucleotide encoding the peptide of interest may be inserted therein. A transformed cell, into which the obtained vector has been incorporated, is incubated after selecting optimal conditions according to the respective host, and then the peptide of interest is recovered and/or purified from the culture medium or the cell itself. Recovery and/or purification are carried out using a property or function of the peptide as an indicator. For example, inhibiting action on the phosphorylation of c-Jun by JNK3 or the phosphorylation by JNK3 can be employed as an indicator. In the case of KIAA1491 complete or the peptide derived therefrom, the phosphorylated protein of KIAA1491 complete or the peptide itself that is phosphorylated by JNK3 can be employed as an indicator. Examples of methods for recovery and/or purification include a technique for fractionation based on solubility difference using ammonium sulfate or alcohol or the like, gel filtration, ion column chromatography, and affinity chromatography. These methods may be used alone or in combination. Preferably, a method is used in which polyclonal antibodies or monoclonal antibodies specific to the peptides are prepared based on information concerning the amino acid sequences of the peptides, and recovery by specific adsorption is conducted using the antibodies.

Antibodies can be prepared using KIAA1491 complete or the peptide derived therefrom as an antigen. The antigen may be a peptide or fragments thereof. The antigen is composed of at least 8, preferably at least 10, more preferably at least 12, and even more preferably 15 or more amino acids. To prepare an antibody specific to KIAA1491 complete or a peptide derived therefrom, it is preferable to use a region comprised of an amino acid sequence specific to KIAA1491 complete or the peptide. The amino acid sequence of the region in question need not necessarily be homologous or identical to the sequence of KIAA1491 complete or the peptide. A site that is exposed to an outside of the tertiary structure thereof is preferable. Even if an amino acid sequence of the exposed site is discontinuous in the primary structure, the sequence is acceptable as long as the amino acid sequence is continuous in the exposed site. The antibody is not particularly limited as long as it immunologically binds to or recognizes the peptide. The presence or absence of the binding or recognition can be determined by known antigen-antibody binding reactions.

Known antibody production methods can be used to produce the antibody. The antibody can be obtained, for example, by inducing immunity such as a humoral response and/or cellular response after administration to an animal of the antigen with or without an adjuvant, by itself or in a condition where it is bound to a carrier. The carrier is not particularly limited, as long as it does not itself exhibit any adverse action to the host and increase the antigenicity. Examples of carriers include cellulose, polymerized amino acids, albumin, and keyhole limpet hemocyanin. Examples of adjuvants include Freund's complete adjuvant (FCA), Freund's incomplete adjuvant (FIA), Ribi (MPL), Ribi (TDM), Ribi (MPL+TDM), Bordetella pertussis vaccine, muramyl dipeptide (MDP), aluminum adjuvant (ALUM), and combinations thereof. As the immunized animal, a mouse, rat, goat, rabbit, horse or the like is preferably used.

A polyclonal antibody can be obtained by well-known antibody collection methods from the serum of an animal that was treated with an immunizing means. Antibody affinity chromatography is a preferred antibody collection method.

A monoclonal antibody can be produced by collecting antibody-producing cells (i.e., spleen or lymph cells derived from a lymph node) from an animal, that was treated with an immunizing means, and then introducing a well-known means for transforming the cells to permanently proliferating cells (for example, using myeloma cells such as P3-X63-Ag8). For example, the antibody producing cell and permanently proliferating cell are fused together by known methods to form a hybridoma. The hybridoma may be then cloned. After selecting, from the cloned hybridomas, a hybridoma that produces an antibody that specifically recognizes the peptide of interest the antibody may be collected from a culture solution of the selected hybridoma.

The thus-obtained polyclonal antibody or monoclonal antibody can be used as an antibody for purification, a reagent, a labeling marker or the like for the peptide.

A pharmaceutical composition that comprises as an active ingredient at least one member of KIAA1491 complete or peptides derived therefrom, a polynucleotide encoding any of these, a vector comprising the polynucleotide, a transformant transfected with the vector, and an antibody that recognizes any of the peptides, are also included within the scope of the present invention.

The present invention provides an effective means for identifying an agent for inhibiting activity or an agent for promoting activity of KIAA1491 complete, or an agent for inhibiting expression or an agent for promoting expression of KIAA1491 complete, by using KIAA1491 complete or the peptide derived therefrom, the polynucleotide comprising the nucleotide sequence encoding KIAA1491 complete or the complementary nucleotide sequence thereof, a vector comprising the polynucleotide, the transformant formed by introduction of the vector, the antibody that immunologically recognizes KIAA1491 complete or the peptide derived therefrom, and the protein synthesis system using them alone or in combination of a plurality thereof. The method for identification can be constructed using well-known screening systems for pharmaceutical preparations. Examples of a test substance include a compound derived from a natural product or a chemical library, or a compound obtained by drug design based on the tertiary structure of KIAA1491 complete or the peptide derived therefrom.

Specifically, a compound that inhibits or promotes the activity of a peptide of the present invention can be identified by selecting conditions that enable specific interaction, such as contact between KIAA1491 complete or the peptide derived therefrom and the test substance, introducing thereto a system using a signal and/or marker that is capable of detecting the presence or absence of the interaction, and then detecting the presence, absence or change of the signal and/or the marker. For example, it is possible to screen for a compound that can regulate the interaction or the phosphorylation, by employing interaction between KIAA1491 complete or the peptide derived therefrom and JNK3, or the phosphorylation by JNK3 as the indicator. Specifically, for example, a test substance is added to coexist with KIAA1491 complete, JNK3 and ATP (adenosine triphosphate) to conduct the phosphorylation, and the resulting amount of phosphorylated KIAA1491 complete is quantified. When the amount of phosphorylated protein decreases in comparison with a result obtained from a reaction to which the test substance was not added, it can be determined that the test substance inhibits interaction between KIAA1491 complete and JNK3. In contrast, when the amount of phosphorylated protein increases, it can be determined that the test substance promotes interaction between KIAA1491 complete and JNK3. As a different example, a method for identification may be mentioned, in which a test substance is added to coexist with KIAA1491 complete, JNK3, c-Jun and ATP to conduct the phosphorylation, and the resulting amount of phosphorylated c-Jun is quantified. When the amount of phosphorylated c-Jun decreases in comparison with a result obtained from a reaction to which the test substance was not added, it can be determined that the test substance promotes interaction between KIAA1491 complete and JNK3. In contrast, when the amount of phosphorylated c-Jun increases, it can be determined that the test substance inhibits interaction between KIAA1491 complete and JNK3.

A compound that promotes or inhibits the expression of the polynucleotide encoding KIAA1491 complete or the peptide derived therefrom can be identified by selecting conditions that enable expression of the polynucleotide and allowing the test substance to be present therein, introducing thereto a system using a signal and/or marker that is capable of detecting the expression of the polynucleotide, and then detecting the presence, absence or change of the signal and/or marker. For example, a compound that influences the expression of a gene of the present invention can be identified by preparing a vector in which a reporter gene is connected in downstream of a promoter region of KIAA1491 complete gene in place of the polynucleotide encoding KIAA1491 complete, then contacting the test substance with a cell, such as an eukaryotic cell into which the vector was introduced, and finally, detecting the presence, absence or change of the reporter gene expression. When the expression amount of the reported gene increases in comparison to a case where the cell was not contacted with the test substance, it can be determined that the test substance promotes the expression of KIAA1491 complete. In contrast, when the expression amount decreases, it can be determined that the test substance inhibits the expression of KIAA1491 complete.

As used herein, the term “signal” refers to a substance that can itself be directly detected by means of the physical or chemical properties thereof, and the term “marker” refers to a substance that can be indirectly detected using the physical or biological properties thereof as an indicator. Examples of signals that can be used herein include known signals such as luciferase, GFP and radioactive isotopes, and examples of markers include known markers such as a reporter gene, for example, β-galactosidase or chloramphenicol acetyl transferase (CAT) gene, or a detection tag such as 6×His-tag. Methods for detecting these signals or markers are known by those skilled in the art.

### EXAMPLES

Hereunder, the present invention is described in detail based on examples. However, the present invention is not limited to the following examples.

### Example 1

(In Silico Search for Proteins that Interact with JNK3)

Prediction in silico of the proteins that interact with JNK3 was conducted according to a prediction method described in International Publication No. WO 01/67299 (Patent Document 1). First, the amino acid sequence of JNK3 was decomposed into oligopeptides having a pre-determined length in order to search in a database for proteins having the amino acid sequences of each of the oligopeptides, or having the homologous amino acid sequences to these amino acid sequences. Next, local alignment between the proteins obtained and JNK3 was conducted based on the prediction that the proteins having a high local alignment score might be those interacting with JNK3. The criterion for the local alignment score was the same as that in the method described in International Publication No. WO 01/67299, which is to say, more than 25.0. Since it is known that JNK3 is a protein that is specifically expressed in the cerebral nervous system and is expressed in a state of shock, such as hypoxia, and imparts damage to cerebral function, the candidate proteins interacting with JNK3 were narrowed down to proteins that are known to be expressed in the brain and are known to have an important function.

As a result, it was found that the oligopeptide QTFDKQ (SEQ ID NO: 12), which has homology to the oligopeptide QGFDKQ (SEQ ID NO: 11) comprising six amino acid residues derived from JNK3, is present in the amino acid sequence of the protein KIAA1491 (SEQ ID NO: 6) with unknown function. FIG. I shows the results of local alignment between JNK3 (the upper sequences in the figure) and KIAA1491 (the lower sequences in the figure).

(In-Silico Determination of Full-Length KIAA1491 Sequence)

The HUGE database of Kazusa DNA Research Institute indicated the possibility that the N-terminus of KIAA1491 (SEQ ID NO: 6) was deleted. Therefore the present inventors carried out an in-silico prediction of the cDNA sequence encoding a region on the N-terminal side. First, 120 bases at the 5′-terminus of KIAA1491 were searched using BLASTN, whereby it was found to have 100% homology with 120 bases of QVI-MT0132-201100-498-b05 MTO132 Homo sapiens cDNA (GenBank accession number: BF894928). Since bases I to 120 of KIAA1491 corresponded to bases 358 to 477 of BF894928, it was considered that BF894928 was an upstream sequence of KIAA1491. Next, by taking as a first methionine the second methionine from upstream obtained by in-silico translation of BF894928 using the same frame as KIAA1491, it was confirmed through RT-PCR using human brain-derived poly-A RNA that the corresponding mRNA was present in brain. The cDNA extending across the upstream region until the methionine was denoted as “KIAA1491 complete cDNA” (SEQ ID NO: 7) and the full-length KIAA1491 that includes the amino acid sequence up to the methionine was denoted as “KIAA1491 complete” (SEQ ID NO: 1).

(Analysis of Action of KIAA1491 Complete on the phosphorylation of c-Jun by JNK3).

Materials

KIAA1491 complete was expressed as an N-terminal GST-fusion protein (hereafter, referred to as “GST-KIAA1491 complete”) in Escherichia coli, and then purified using Glutathione Sepharose 4B (Amersham Pharmacia Biotech) in preparation for use.

Specifically, after amplifying the ORF region of KIAA1491 complete cDNA by PCR using cDNA from human brain as a template, the amplification product was integrated into a pDEST15 vector (Invitrogen Corp.) using the Gateway Cloning Technology (Invitrogen Corp.) cloning system to construct a GST-KIAA1491 complete expression vector for Escherichia coli.

After introducing the vector into E. coli BL21-SI strain (Invitrogen) and culturing at 37° C. in LBON medium (LB medium without NaCl) containing 100 μg/ml of ampicillin, NaCl was added thereto to bring to a final concentration of 0.3 M, followed by further culturing at 25° C. to induce expression of GST-KIAA1491 complete. The bacterial cells were recovered and an extract was prepared, and then the GST-KIAA1491 complete was purified using Glutathione Sepharose 4B. The purified protein was dialyzed in preparation for use.

Activated human JNK3 was prepared as a protein with an N-terminal histidine-tag (His-tag) (hereunder, referred to as “His-JNK3”). First, human JNK3 (JNK3α1) cDNA, obtained by RT-PCR using a human hippocampus cDNA library as a template, was inserted into pFastBac HT (Invitrogen Corp.) to produce a recombinant baculovirus for expression of His-JNK3, as instructed by the manufacturer. Next, Sf9 cells were infected with the recombinant virus to express His-JNK3, and the His-JNK3 was purified with Probond Resin (Invitrogen Corp.) and used.

In an in vitro kinase assay of c-Jun, a peptide (c-Jun (1-79)) comprising a 79-amino acid region at the N-terminus of c-Jun was used in place of c-Jun. The c-Jun (1-79) was expressed as an N-terminal GST fusion protein (hereunder, referred to as “GST-c-Jun (1-79)”) in E. Coli, and then purified using Glutathione Sepharose 4B (Amersham Pharmacia Biotech) in preparation for use.

In Vitro Kinase Assay

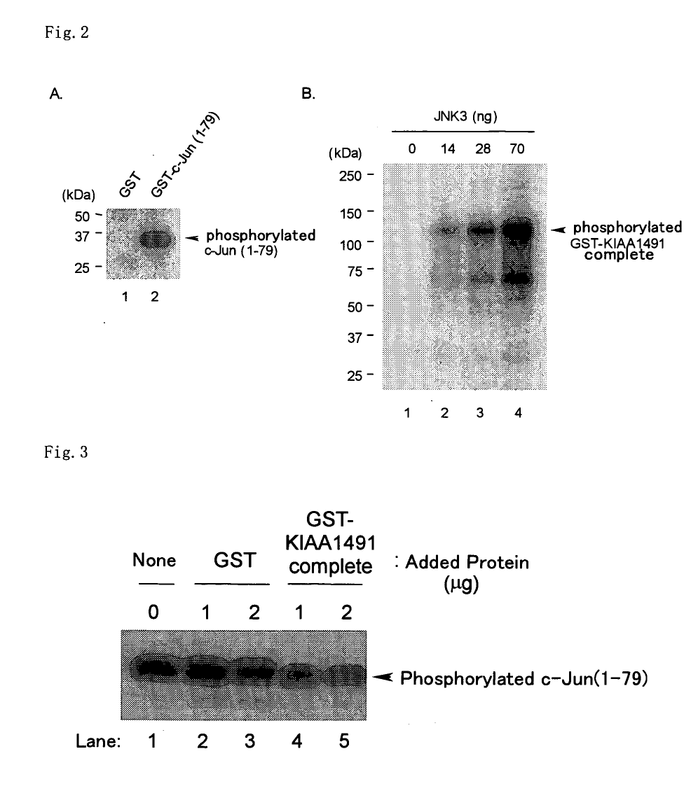

The phosphorylation reaction was performed by incubating 1 μg of each of the GST fusion proteins (GST-KIAA1491 complete, GST-c-Jun (1-79), or GST) and activated JNK3 (0, 14, 28, or 70 ng) in a kinase buffer (25 mM Tris-HCl, pH 7.5/5 mM β-glycerophosphate/2 mM dithiothreitol (DTT)/0.1 mM Na3VO4/10 mM MgCl2/10 μM ATP) containing 5 μCi of [γ-32P] ATP (3000 Ci/mmol, NEN Life Science Products) at 30° C. for 30 min. After the reaction, an equivalent amount of 2×SDS sample buffer (4% SDS/125 mM Tris-HCl, pH6.8/20% glycerol/0.01% bromophenol blue (BPB)/10% β-mercaptoethanol) was added and treated for 5 min at 100° C., and the protein was then separated using 5-20% SDS-PAGE to detect the phosphorylated protein by autoradiography using BAS2000 (Fuji Photo Film Co., Ltd.) (FIG. 2A and FIG. 2B).

Next, the phosphorylation reaction was performed by incubating 1 μg of GST-c-Jun (1-79) and activated JNK3 (70 ng) in a kinase buffer containing 100 μM of ATP at 30° C. for 20 min in the presence or absence of GST or GST-KIAA1491 complete (both consisting of 1 μg or 2 μg). After the reaction, an equivalent amount of 2×SDS sample buffer was added, the resulting mixture was treated for 5 min at 100° C., the supernatant was separated using 10% SDS-PAGE, and phosphorylated GST-c-Jun (1-79) was detected by immunoblotting using anti-Phospho-c-Jun (S63) antibody (New England Biolabs Inc.) as the primary antibody and HRP-conjugated anti-rabbit IgG antibody (Amersham Pharmacia Biotech, Inc.) as the secondary antibody (FIG. 3). An ECL Western Blotting Detection Kit (Amersham Pharmacia Biotech, Inc.) was used for the detection. The band strength of the detected phosphorylated GST-c-Jun (1-79) was quantified using image analysis software, Intelligent Quantifier (Bio Image Inc.), and the band strengths in the presence of GST or GST-KIAA1491 complete were shown as relative strengths when the strength in the absence of GST and GST-KIAA1491 complete was taken as 100% (Table 1).

Results

c-Jun and GST-KIAA1491 complete were phosphorylated by JNK3 (FIG. 2A and FIG. 2B). Since the phosphorylation was dependent on the dose of JNK3 (FIG. 2B), it was clarified that the phosphorylation of GST-KIAA1491 complete was not self-phosphorylation, but was caused by JNK3. It was thus determined that JNK3 and KIAA1491 complete interact with each other.

Further, as shown in FIG. 3 and Table 1, in the presence of 1 μg or 2 μg of GST-KIAA1491 complete, the phosphorylation of GST-c-Jun (1-79) by JNK3 was inhibited approximately 30%. In contrast, in the presence of GST as a negative control, there was no change in the phosphorylation of GST-c-Jun (1-79) by JNK3. It was thus clarified that the phosphorylation of GST-c-Jun (1-79) by JNK3 is inhibited by KIAA1491 complete.

TABLE 1

| Added Protein | Added Amount (μg) | Relative Strength (%) |
|---|---|---|
| None | | 100 |
| GST | 1.0 | 92.7 |
| | 2.0 | 93.6 |
| GST-KIAA1491 complete | 1.0 | 71.5 |
| | 2.0 | 67.1 |

### Example 2

(In-Silico Search for Proteins that Interact with JNK3)

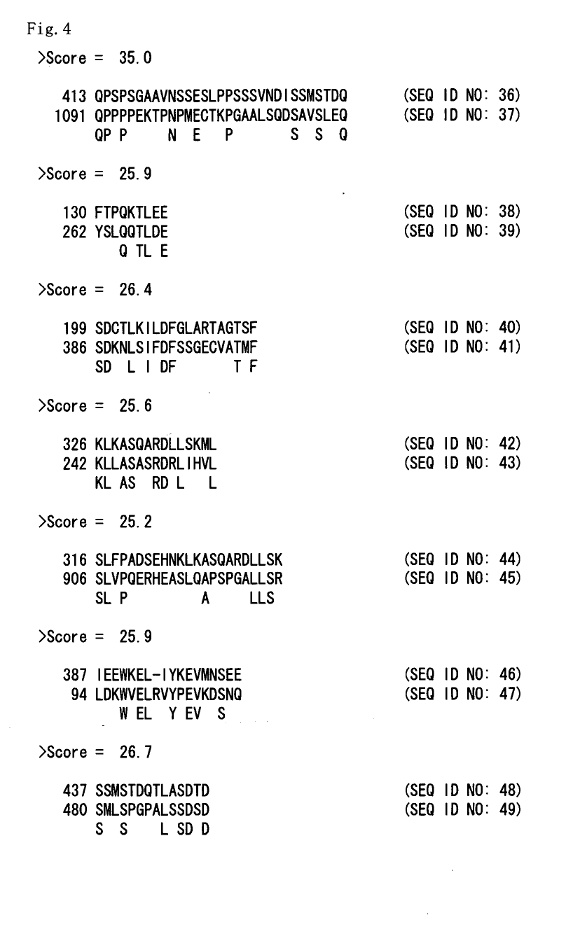

Predictions of the proteins that interact with JNK3 were conducted in silico in the same manner as in Example 1. As a result, it was found that the oligopeptide SLPPAD (SEQ ID NO: 14), which has homology with the oligopeptide SLFPAD (SEQ ID NO: 13) comprising six amino acid residues derived from JNK3 is present in the amino acid sequence of a protein KIAA0596 (GenBank accession number: ABO 11168) with unknown function. FIG. 4 shows the results of local alignment between JNK3 and KIAA0596.

(Analysis of Zction of KIAA0596 on the phosphorylation of c-Jun by JNK3)

It was clarified that KIAA0596 exhibits a homology of 74% to mouse JNK-binding protein 1 (JNKBP1, Non-patent Document 4) (GenBank accession number: AB029482), which is reported as binding with JNK3. It is known that the C-terminal region of mouse JNKBP 1 is required for the binding thereof to JNK3. Therefore, binding between a C-terminal region of KIAA0596 (amino acid sequence from amino acid 639 to 1217, hereafter referred to as “KIAA0596CT”) and JNK3 was analyzed by a far-Western method. Further, the action of KIAA0596CT on the phosphorylation of c-Jun by activated JNK3 was assayed.

Materials

The human JNK3 used in the binding assay was prepared as a fusion protein (MBP-JNK3) with an N-terminal maltose binding protein. First, human JNK3 (JNK3α1) cDNA, obtained by RT-PCR using a human hippocampus cDNA library as a template, was inserted into pMAL-c2 (New England Biolabs Inc.) to construct an MBP-JNK3 expression vector as instructed by the manufacturer. The vector was introduced into E. Coli DH5a strain, and expression of MBP-JNK3 was induced. Bacterial cells were recovered and the extract was prepared, and then the MBP-JNK3 was purified using amylose resin. The purified protein was dialyzed in preparation for use.

The activated human JNK3 used in the kinase assay was prepared as a protein with an N-terminal His-tag (hereunder, referred to as “His-JNK3”) in the same manner as in Example 1.

Human KIAA0596CT was expressed in E. coli as an N-terminal GST fusion protein (hereunder, referred to as “GST-KIAA0596CT”) and then purified with Glutathione Sepharose 4B (Amersham Pharmacia Biotech, Inc.) in preparation for use.

Specifically, first, a region from nucleotide 1916 to 3655 in the nucleotide sequence of KIAA0596 was amplified by PCR using a KIAA0596 clone (purchased from Kazusa DNA Research Institute) as a template. Next, the amplification product was integrated into a pDEST15 vector (Invitrogen Corp.) using Gateway Cloning Technology (Invitrogen Corp.) to construct a GST-KIAA0596CT expression vector for E. coli.

After introducing the vector into E. coli BL21-SI strain (Invitrogen Corp.) and culturing at 37° C. in LBON medium (LB medium without NaCl) containing 100 μg/ml of ampicillin, NaCl was added thereto to a final concentration of 0.3 M, followed by further culturing at 25° C. to induce expression of GST-KIAA0596CT. Bacterial cells were recovered and the extract was prepared, and then the GST-KIAA0596CT was purified using Glutathione Sepharose 4B. The purified protein was dialyzed in preparation for use.

Binding Assay

After spotting 1 μg of GST or GST-KIAA0596CT on nitrocellulose membranes (Schleicher & Schuell BioScience, BA85), the membranes were incubated at room temperature in TBST (10 mM Tris-HCl, pH 7.5/0.15M NaCl/0.05% Tween 20) containing 5% skim milk. After rinsing the membranes with TBST, they were incubated overnight at 4° C. in TBST containing 1.0 μg/ml of MBP-JNK3 or MBP. After washing the membranes with TBST, bound MBP-JNK3 was detected by immunoblotting using anti-MBP antiserum (New England Biolabs Inc.) as the primary antibody, and HRP-conjugated anti-rabbit IgG antibody (Amersham Pharmacia Biotech, Inc.) as the secondary antibody (FIG. 5). An ECL Western Blotting Detection Kit (Amersham Pharmacia Biotech, Inc.) was used for the detection.

In Vitro Kinase Assay

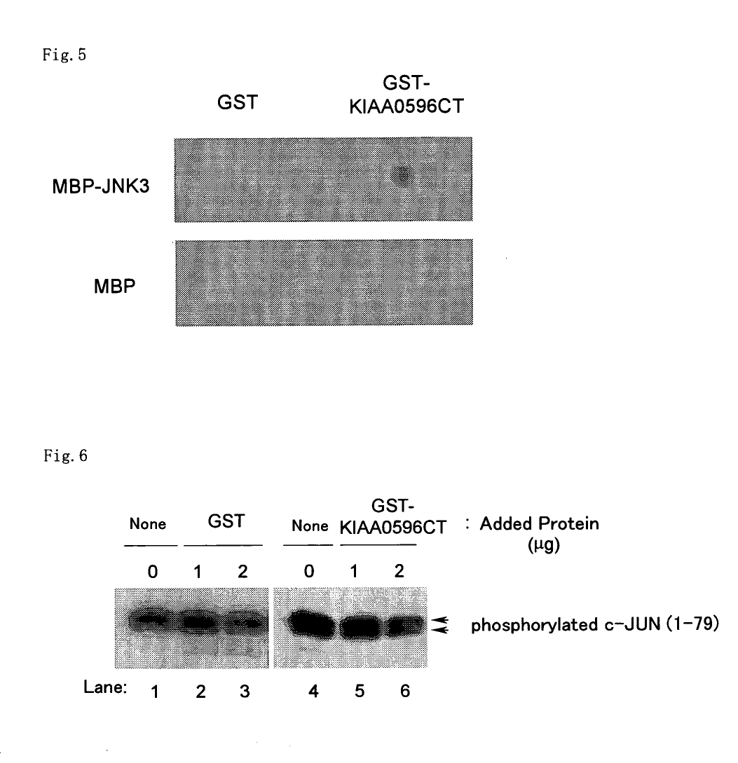

The phosphorylation reaction was performed by incubating 1 μg of GST-c-Jun (1-79) and activated JNK3 (70 ng) in a kinase buffer (25 mM Tris-HCl, pH 7.5/5 mM β-glycerophosphate/2 mM DTT/0.1 mM Na3VO4/10 mM MgCl2) containing 100 μM of ATP at 30° C. for 30 min in the presence or absence of GST or GST-KIAA0596CT (both consisting of 1 μg or 2 μg). After the reaction, an equivalent amount of 2×SDS sample buffer (4% SDS/125 mM Tris-HCl, pH 6.8/20% glycerol/0.01% BPB/10% β-mercaptoethanol) was added and treated for 5 min at 100° C., the supernatant was separated using 10% SDS-PAGE, and phosphorylated GST-c-Jun (1-79) was then detected by immunoblotting using anti-Phospho-c-Jun (S63) antibody (New England Biolabs Inc.) as the primary antibody, and HRP-conjugated anti-rabbit IgG antibody (Amersham Pharmacia Biotech, Inc.) as the secondary antibody (FIG. 6). An ECL Western Blotting Detection Kit (Amersham Pharmacia Biotech, Inc.) was used for the detection. The band strength of the detected phosphorylated GST-c-Jun (1-79) was quantified using image analysis software, Intelligent Quantifier (Bio Image Inc.), and the band strengths in the presence of GST or GST-KIAA0596CT were shown as relative strengths when the strength in the absence of GST and GST-KIAA0596CT was taken as 100% (Table 2).

Results

As shown in FIG. 5, binding was observed between GST-KIAA0596CT and MBP-JNK3. Since binding between GST-KIAA0596CT and MBP and binding between GST and MBP-JNK3 was not observed, it was confirmed that the detected binding between GST-KIAA0596CT and MBP-JNK3 was specific.

Further, as shown in FIG. 6 and Table 2, in the presence of 1 μg and 2 μg of KIAA0596CT, the phosphorylation of GST-c-Jun (1-79) by JNK3 was inhibited by approximately 30% and 67%, respectively. In contrast, in the presence of GST as a negative control, there was no change in the phosphorylation of GST-c-Jun (1-79) by JNK3. It was thus clarified that the phosphorylation of GST-c-Jun (1-79) by JNK3 is dose-dependently inhibited by KIAA0596CT.

TABLE 2

| Added Protein | Added Amount (μg) | Relative Strength (%) |
|---|---|---|
| None | | 100 |
| GST | 1.0 | 92.7 |
| | 2.0 | 93.6 |
| GST-KIAA0596CT | 1.0 | 70.6 |
| | 2.0 | 33.5 |

### Example 3

(In-Silico Search for Proteins that Interact with JNK3)

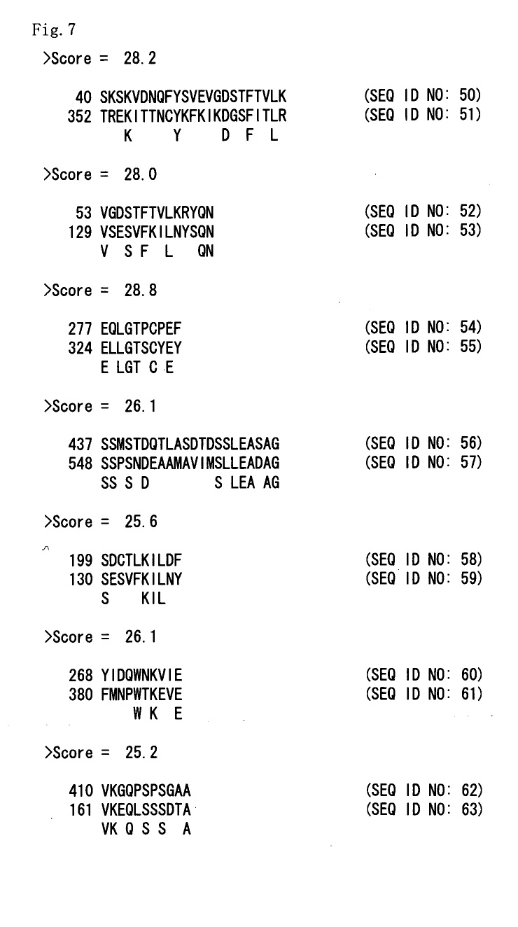

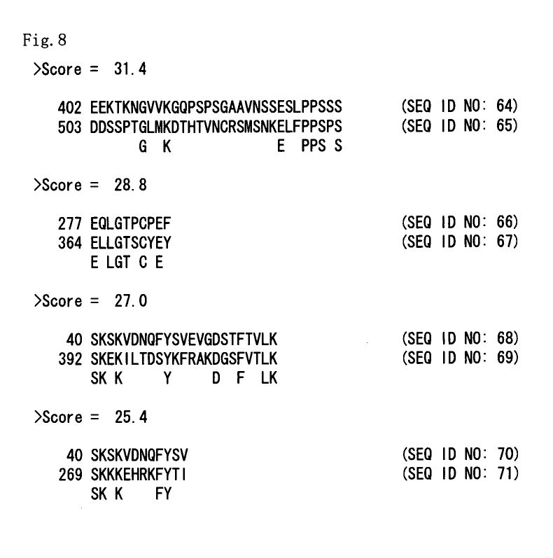

Prediction in silico of proteins that interact with JNK3 was conducted in the same manner as in Example 1. As a result, it was found that the oligopeptide KVKEQL (SEQ ID NO: 16), which has homology to the oligopeptide KVIEQL (SEQ ID NO: 15) comprising six amino acid residues derived from JNK3, is present in the amino acid sequence of a protein, BMAL1, that is associated with circadian rhythm. In addition, it was found that the oligopeptide KVKEQL (SEQ ID NO: 16) is also present in the amino acid sequence of BMAL2, a homologous protein of BMAL1. FIGS. 7 and 8 show the results of local alignment between JNK3 and BMAL1, and JNK3 and BMAL2. Fragments exhibiting a score of more than 25.0 were found in 7 cases in the local alignment between JNK3 and BMAL1, and in 4 cases between JNK3 and BMAL2. It was thus predicted that JNK3 is a protein that interacts more strongly with BMAL1 than with BMAL2.

(Analysis of Action of BMAL1 on the phosphorylation of c-Jun by JNK3)

Materials

Activated human JNK3 was prepared as a protein with an N-terminal histidine-tag (His-JNK3) in the same manner as in Example 1.

c-Jun (1-79) was prepared as an N-terminal GST fusion protein (hereunder, referred to as “GST-c-Jun (1-79)”) in the same manner as in Example 1.

Human BMAL1 was expressed in E. coli as an N-terminal GST fusion protein (hereunder, referred to as “GST-BMAL1”) and then purified using Glutathione Sepharose 4B (Amersham Pharmacia Biotech) in preparation for use. In this example, BMAL1b (SEQ ID NO: 4) was used as the BMAL1.

More specifically, after amplifying by PCR the ORF region of BMAL1 from pcDNA3.1-BMAL1/V5-His (Invitrogen Corp.), which is a human BMAL1 expression plasmid with a C-terminal V5/His-tag added, the amplified product was inserted into pGEX-4T (Amersham Pharmacia Biotech, Inc.) to construct a GST-BMAL1 expression vector for E. coli. The obtained nucleotide sequence (SEQ ID NO: 9) was confirmed by direct sequencing according to a common method.

Next, after culturing E. coli BL21 strain, into which the above expression vector was introduced, at 37° C. in LB medium containing 100 μg/ml of ampicillin, isopropyl-1-thio-β-D-galactopyronoside (IPTG) was added to a final concentration of 0.3 mM, followed by further culturing at 25° C. before recovering bacterial cells. The extract was prepared from these bacterial cells using TGEDS buffer (50 mM Tris-HCl, pH 8.0/0.2 mM ethylenediaminetetraacetic acid (EDTA)/1 mM DTT/150 mM NaCl/10 % glycerol) containing a protease inhibitor cocktail, and the GST-BMAL1 was purified using Glutathione Sepharose 4B. The purified GST-BMAL1 was used after dialysis with TGEDS buffer/0.1% Triton X-100.

In Vitro Kinase Assay

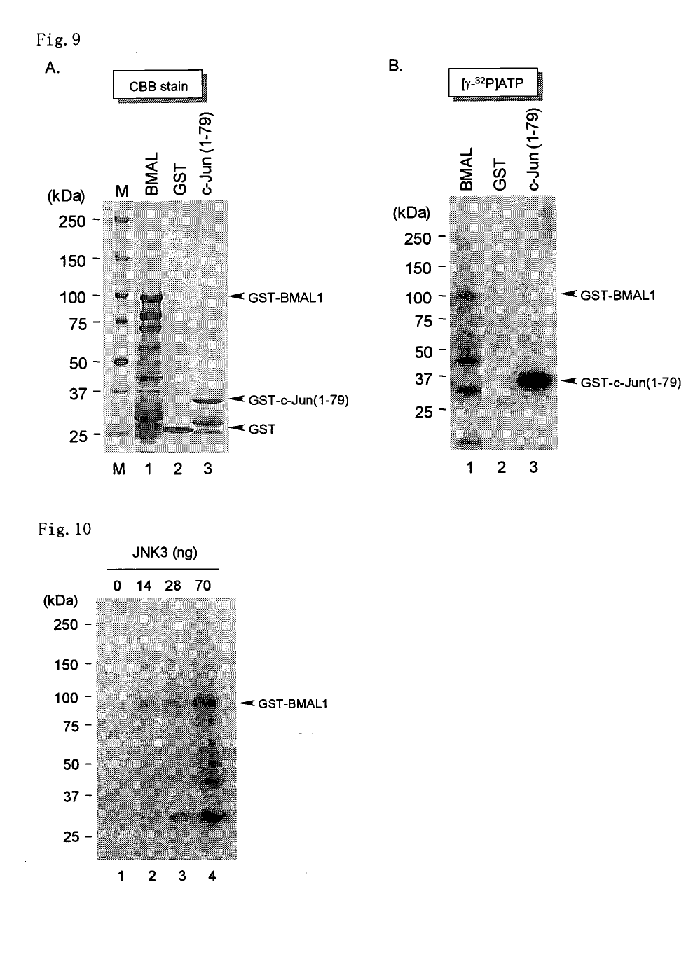

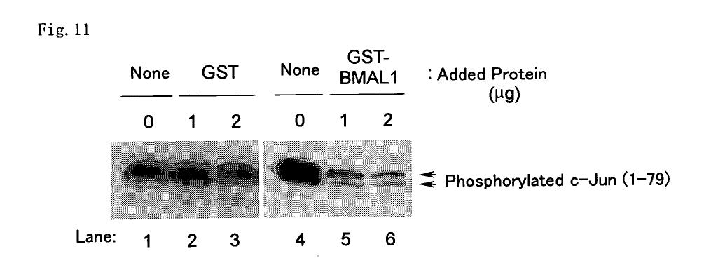

The phosphorylation reaction was performed by incubating 1 μg of each of the above GST fusion proteins (GST-BMAL1, GST-c-Jun (1-79), or GST) and activated JNK3 (70 ng) in a kinase buffer (25 mM Tris-HCl, pH 7.5/5 mM β-glycerophosphate/2 mM DTT/0.1 mM Na3VO4/10 mM MgCl2/10 FM ATP) containing 5 μCi of [γ-32P] ATP (3000 Ci/mmol, NEN Life Science Products) at 30° C. for 30 min. After the reaction, an equivalent amount of 2×SDS sample buffer (4% SDS/125 mM Tris-HCl, pH 6.8/20% glycerol/0.01% BPB/10% β-mercaptoethanol) was added and treated for 5 min at 100° C., and the protein was then separated by 5-20% SDS-PAGE Phosphorylated protein was detected by autoradiography using BAS2000 (Fuji Photo Film Co., Ltd.) (FIG. 9B). Further, after separating each of the used GST fusion proteins (1 μg) by 5-20% SDS-PAGE, the mobility of the proteins of interest was confirmed by CBB staining (FIG. 9A). In order to confirm JNK3 dose-dependency in the phosphorylation reaction of GST-BMAL1, activated JNK3 was added to the reaction system in an amount of 0 ng, 14 ng, 28 ng or 70 ng, and phosphorylated protein was then detected by a similar method (FIG. 10).

Next, the phosphorylation reaction was performed by incubating 1 μg of GST-c-Jun (1-79) and activated JNK3 (70 ng) in a kinase buffer containing 100 μM of ATP at 30° C. for 20 min in the presence or absence of GST or GST-BMAL1 (both consisting of 1 μg or 2 μg). After the reaction, the reaction product was treated in a sample buffer in the same manner as described above, the supernatant was separated by 10% SDS-PAGE, and phosphorylated GST-c-Jun (1-79) was detected by immunoblotting using anti-Phospho-c-Jun antibody (New England Biolabs Inc.) as the primary antibody, and HRP-conjugated anti-rabbit IgG antibody (Amersham Pharmacia Biotech, Inc.) as the secondary antibody (FIG. 11). An ECL Western Blotting Detection Kit (Amersham Pharmacia Biotech, Inc.) was used for the detection. The band strength of the detected phosphorylated GST-c-Jun (1-79) was quantified using image analysis software, Intelligent Quantifier (Bio Image Inc.), and the band strengths in the presence of GST or GST-BMAL1 were shown as relative strengths when the strength in the absence thereof was taken as 100% (Table 3).

Results

As shown in FIG. 9B, the phosphorylation of GST-c-Jun (1-79) by JNK3 was observed. Since phosphorylation was not observed in the GST as a negative control, it was confirmed that the assay system used in this example is appropriate for measuring JNK3 activity. In this assay system, the phosphorylation of GST-BMAL1 by JNK3 was observed. Further, since the phosphorylation was dependent on the dose of JNK3, it was clarified that the phosphorylation of GST-BMAL1 was not self-phosphorylation, but was caused by JNK3 (FIG. 10). It was thus determined that JNK3 and BMAL1 interact each other.

In addition, as shown in FIG. 11 and Table 3, in the presence of 1 μg and 2 μg of GST-BMAL1, phosphorylation of GST-c-Jun (1-79) by JNK3 decreased to as far as approximately 15% and 5%, respectively. In contrast, there was no change in the phosphorylation of GST-c-Jun (1-79) by JNK3 in the presence of GST. It was thus clarified that the phosphorylation of GST-c-Jun (1-79) by JNK3 is inhibited by BMAL1.

TABLE 3

| Added Protein | Added Amount (μg) | Relative Strength (%) |
|---|---|---|
| None | | 100 |
| GST | 1.0 | 92.7 |
| | 2.0 | 93.6 |
| GST-BMAL1 | 1.0 | 15.3 |
| | 2.0 | 4.7 |

### Example 4

(In-Silico Search for Proteins that Interact with JNK3)

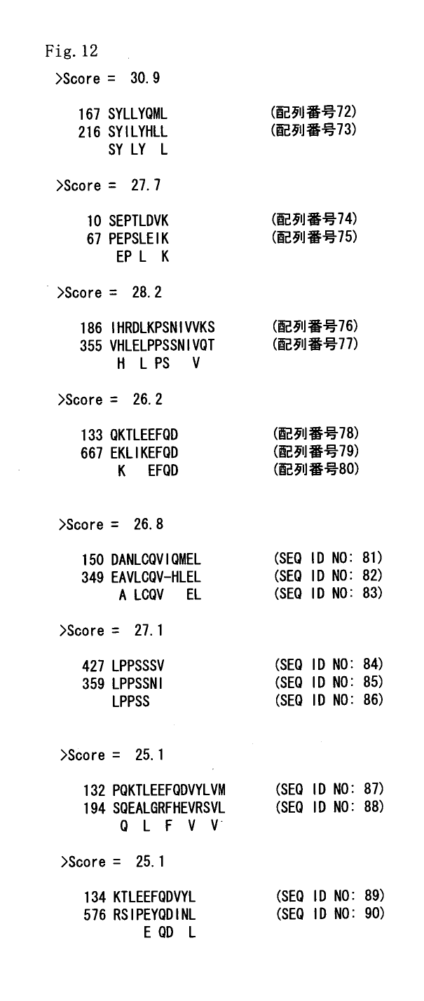

Prediction in silico of proteins that interact with JNK3 was conducted in the same manner as in Example 1. As a result, it was found that oligopeptides LPPSSN (SEQ ID NO: 19) and AVLCQV (SEQ ID NO: 20), which have homology to oligopeptides LPPSSS (SEQ ID NO: 17) and ANLCQV (SEQ ID NO: 18) comprising six amino acid residues derived from JNK3, are present in the amino acid sequence of BPL1, which is a holocarboxylase synthetase. FIG. 12 shows the results of local alignment between JNK3 and BPL1. Eight fragments exhibiting a score of more than 25.0 were found for the local alignment between JNK3 and BPL1. It was thus predicted that JNK3 and BPL1 are interacting proteins.

(Analysis of Action of BPL1 on the phosphorylation of c-Jun by JNK3)

Materials

Activated human JNK3 was prepared as a protein with an N-terminal histidine-tag (His-JNK3) in the same manner as in Example 1.

c-Jun (1-79) was prepared as an N-terminal GST fusion protein (hereunder, referred to as “GST-c-Jun (1-79)”) in the same manner as in Example 1.

Human BPL1 was expressed as an N-terminal GST fusion protein (hereafter, referred to as “GST-BPL1”) in E. coli, and then purified using Glutathione Sepharose 4B (Amersham Pharmacia Biotech, Inc.) in preparation for use. In this example, a peptide comprised of the amino acid sequence described in SEQ ID NO: 5 was used as the BPL1.

More specifically, after amplifying by PCR the ORF region of BPL1 from pcDNA3.1-BPL1/V5-His (Invitrogen Corp.), which is a human BPL1 expression plasmid with the C-terminal V5/His-tag added, the amplified product was inserted into pGEX-4T (Amersham Pharmacia Biotech, Inc.) to construct a GST-BPL1 expression vector for E. coli. The obtained nucleotide sequence (SEQ ID NO: 10) was confirmed by direct sequencing according to a common method.

Next, after culturing E. coli BL21 strain into which the above expression vector was introduced, at 37° C. in LB medium containing 100 μg/ml of ampicillin, IPTG was added thereto to a final concentration of 0.1 mM, followed by further culturing at 25° C. before recovering bacterial cells. An extract was prepared from these bacterial cells using TGEDS buffer (50 mM Tris-HCl, pH 8.0/0.2 mM EDTA/1 mM DTT/150 mM NaCl/10% glycerol) containing a protease inhibitor cocktail, and the GST-BPL1 was purified using Glutathione Sepharose 4B. The purified GST-BPL1 was dialyzed using a TGEDS buffer in preparation for use.

In Vitro Kinase Assay

The phosphorylation reaction was performed by incubating 1 μg of each GST fusion protein (GST-BPL1, GST-c-Jun (1-79), or GST) and activated JNK3 (0, 14, 28 or 70 ng) in a kinase buffer (25 mM Tris-HCl, pH 7.5/5 mM glycerophosphate/2 mM DTT/0.1 mM Na3VO4/10 mM MgCl2/10 μM ATP) containing 5 μCi of [γ-32P] ATP (3000 Ci/mmol, NEN Life Science Products) at 30° C. for 30 min. After the reaction, an equivalent amount of 2×SDS sample buffer (4% SDS/125 mM Tris-HCl, pH 6.8/20% glycerol/0.01% BPB/10% β-mercaptoethanol) was added and treated for 5 min at 100° C., and the protein was then separated by 5-20% SDS-PAGE. Phosphorylated protein was detected by autoradiography using BAS2000 (Fuji Photo Film Co., Ltd.).

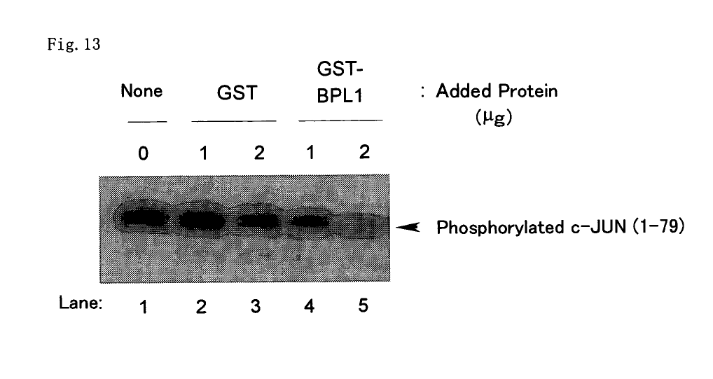

Next, the phosphorylation reaction was conducted by incubating 1 μg of GST-c-Jun (1-79) and activated JNK3 (70 ng) in a kinase buffer containing 5 μCi of [γ-32P] ATP (3000 Ci/mmol, NEN Life Science Products) at 30° C. for 30 min in the presence or absence of GST or GST-BPL1 (both consisting of 1 μg or 2 μg). After the reaction, an equivalent amount of 2×SDS sample buffer was added, and after treatment for 5 min at 100° C., the supernatant was separated by 10% SDS-PAGE, and the phosphorylated GST-c-Jun (1-79) was detected by immunoblotting using anti-Phospho-c-Jun (S63) antibody (New England Biolabs Inc.) as the primary antibody, and HRP-conjugated anti-rabbit IgG antibody (Amersham Pharmacia Biotech, Inc.) as the secondary antibody (FIG. 13). An ECL Western Blotting Detection Kit (Amersham Pharmacia Biotech, Inc.) was used for the detection. The band strength of the detected phosphorylated GST-c-Jun (1-79) was quantified using image analysis software, Intelligent Quantifier (Bio Image Inc.), and the band strengths in the presence of GST or GST-BPL1 were shown as relative strengths when the strength in the absence thereof was taken as 100% (Table 3).

Results

The phosphorylation of GST-BPL1 by JNK3 was observed. Since the phosphorylation was dependent on the dose of JNK3, it was clarified that the phosphorylation of GST-BPL1 was not self-phosphorylation, but was caused by JNK3. It was thus determined that JNK3 and BPL1 interact with each other.

As shown in FIG. 13 and Table 4, in the presence of 1 μg and 2 μg of GST-BPL1, the phosphorylation of GST-c-Jun (1-79) by JNK3 was inhibited by approximately 20% and 40%, respectively. In contrast, in the presence of GST, there was no change in the phosphorylation of GST-c-Jun (1-79) by JNK3. It was thus clarified that the phosphorylation of GST-c-Jun (1-79) by JNK3 is dose-dependently inhibited by BPL1.

TABLE 4

| Added Protein | Added Amount (μg) | Relative Strength (%) |
|---|---|---|
| None | | 100 |
| GST | 1.0 | 92.7 |
| | 2.0 | 93.6 |
| GST-BPL1 | 1.0 | 81.6 |
| | 2.0 | 59.7 |

Possibilities for Industrial Use

The present invention provides KIAA1491 complete (SEQ ID NO:1), KIAA0596CT (SEQ ID NO:2), KIAA0596 (SEQ ID NO:3), BMAL1, and BPL1 which have a function for interacting with JNK3, and hence allows utilizing these peptides to inhibit the phosphorylation of c-Jun by JNK3. c-Jun is known to be a transactivator that has a transcription activation activity. Additionally, JNK3 is known to play a role in diseases that involve neurocyte death, such as polyglutamine disease and Alzheimer's disease. Accordingly, it is anticipated that one or more of KIAA1491 complete (SEQ ID NO:1), KIAA0596CT (SEQ ID NO:2), KIAA0596 (SEQ ID NO:3), BMAL1, and BPL1, which have a function for interacting with JNK3, can be used to inhibit the phosphorylation of c-Jun by JNK3, so that the ability to activate transcription of c-Jun may be suppressed and further, so that a physiological phenomenon caused by the phosphorylation of c-Jun by JNK3, such as apoptosis, may be suppressed.

The present invention allows utilizing KIAA1491 complete (SEQ ID NO:1), KIAA0596CT (SEQ ID NO:2), KIAA0596 (SEQ ID NO:3), BMAL1, and BPL1, which have a function for interacting with JNK3, as well as a peptide derived therefrom to inhibit the phosphorylation and functions of c-Jun (such as transcription activation activity), resulting in suppressing apoptosis (such as preventing neurocyte apoptosis). Thus, the present invention also can provide a drug, a pharmaceutical composition and a method for use in preventing and/or treating a neurodegenerative disease (such as polyglutamine disease and Alzheimer's disease). The present invention is also very useful for researching the JNK signaling pathway and its function.

Free Text in Sequencing Listing

SEQ ID NO:11: Partial oligopeptide of JNK3 showing a high score in the local alignment between JNK3 and KIAA1491 (SEQ ID NO:6).

SEQ ID NO:12: Partial oligopeptide of KIAA1491 showing a high score in the local alignment between JNK3 and KIAA1491 (SEQ ID NO:6).

SEQ ID NO:13: Peptide comprised of 6 consecutive amino acid residues in the amino acid sequence of JNK3.

SEQ ID NO:14: Partial peptide of KIAA0596 (SEQ ID NO:3) that has high homology to a peptide comprised of the amino acid sequence represented by SEQ ID NO:9.

SEQ ID NO:15: Peptide comprised of 6 consecutive amino acid residues in the amino acid sequence of JNK3.

SEQ ID NO:16: Partial peptide of BMAL1 or BMAL2 that has high homology to a peptide comprised of the amino acid sequence represented by SEQ ID NO: 11.

SEQ ID NO:17: Peptide comprised of 6 consecutive amino acid residues in the amino acid sequence of JNK3.

SEQ ID NO:18: Peptide comprised of 6 consecutive amino acid residues in the amino acid sequence of JNK3.

SEQ ID NO:19: Partial peptide of BPL1 that has a high homology to a peptide comprised of the amino acid sequence represented by SEQ ID NO: 13.

SEQ ID NO:20: Partial peptide of BPL1 that has a high homology to a peptide comprised of the amino acid sequence represented by SEQ ID NO: 14.

SEQ ID NO:21: Partial oligopeptide of JNK3 showing a high score in the local alignment between JNK3 and KIAA1491 (SEQ ID NO:6).

SEQ ID NO:22: Partial oligopeptide of KIAA1491 showing a high score in the local alignment between JNK3 and KIAA1 491 (SEQ ID NO:6).

SEQ ID NO:23: Partial oligopeptide of JNK3 showing a high score in the local alignment between JNK3 and KIAA1491 (SEQ ID NO:6).

SEQ ID NO:24: Partial oligopeptide of KIAA1491 showing a high score in the local alignment between JNK3 and KIAA1491 (SEQ ID NO:6).

SEQ ID NO:25: Partial sequence which is common in sequence between JNK3 and KIAA1491 (SEQ ID NO:6).

SEQ ID NO:26: Partial oligopeptide of JNK3 showing a high score in the local alignment between JNK3 and KIAA1491 (SEQ ID NO:6).

SEQ ID NO:27: Partial oligopeptide of KIAA1491 showing a high score in the local alignment between JNK3 and KIAA1491 (SEQ ID NO:6).

SEQ ID NO:28: Partial sequence which is common in sequence between JNK3 and KIAA1491 (SEQ ID NO:6).

SEQ ID NO:29: Partial oligopeptide of JNK3 showing a high score in the local alignment between JNK3 and KIAA1491 (SEQ ID NO:6).

SEQ ID NO:30: Partial oligopeptide of KIAA1491 showing a high score in the local alignment between JNK3 and KIAA1491 (SEQ ID NO:6).

SEQ ID NO:3 1: Partial sequence which is common in sequence between JNK3 and KIAA1491 (SEQ ID NO:6).

SEQ ID NO:32: Partial oligopeptide of JNK3 showing a high score in the local alignment between JNK3 and KIAA1491 (SEQ ID NO:6).

SEQ ID NO:33: Partial oligopeptide of KIAA1491 showing a high score in the local alignment between JNK3 and KIAA1491 (SEQ ID NO:6).

SEQ ID NO:34: Partial oligopeptide of JNK3 showing a high score in the local alignment between JNK3 and KIAA1491 (SEQ ID NO:6).

SEQ ID NO:35: Partial oligopeptide of KIAA1491 showing high score in the local alignment between JNK3 and KIAA1491 (SEQ ID NO:6).

SEQ ID NO:36: Partial oligopeptide of JNK3 showing a high score in the local alignment between JNK3 and KIAA0596 (SEQ ID NO:3).

SEQ ID NO:37: Partial oligopeptide of KIAA0596 showing a high score in the local alignment between JNK3 and KIAA0596 (SEQ ID NO:3).

SEQ ID NO:38: Partial oligopeptide of JNK3 showing a high score in the local alignment between JNK3 and KIAA0596 (SEQ ID NO:3).

SEQ ID NO:39: Partial oligopeptide of KIAA0596 showing a high score in the local alignment between JNK3 and KIAA0596 (SEQ ID NO:3).

SEQ ID NO:40: Partial oligopeptide of JNK3 showing a high score in the local alignment between JNK3 and KIAA0596 (SEQ ID NO:3).

SEQ ID NO:41: Partial oligopeptide of KIAA0596 showing a high score in the local alignment between JNK3 and KIAA0596 (SEQ ID NO:3).

SEQ ID NO:42: Partial oligopeptide of JNK3 showing a high score in the local alignment between JNK3 and KIAA0596 (SEQ ID NO:3).

SEQ ID NO:43: Partial oligopeptide of KIAA0596 showing a high score in the local alignment between JNK3 and KIAA0596 (SEQ ID NO:3).

SEQ ID NO:44: Partial oligopeptide of JNK3 showing a high score in the local alignment between JNK3 and KIAA0596 (SEQ ID NO:3).

SEQ ID NO:45: Partial oligopeptide of KIAA0596 showing a high score in the local alignment between JNK3 and KIAA0596 (SEQ ID NO:3).

SEQ ID NO:46: Partial oligopeptide of JNK3 showing a high score in the local alignment between JNK3 and KIAA0596 (SEQ ID NO:3).

SEQ ID NO:47: Partial oligopeptide of KIAA0596 showing a high score in the local alignment between JNK3 and KIAA0596 (SEQ ID NO:3).

SEQ ID NO:48: Partial oligopeptide of JNK3 showing a high score in the local alignment between JNK3 and KIAA0596 (SEQ ID NO:3).

SEQ ID NO:49: Partial oligopeptide of KIAA0596 showing a high score in the local alignment between JNK3 and KIAA0596 (SEQ ID NO:3).

SEQ ID NO:50: Partial oligopeptide of JNK3 showing a high score in the local alignment between JNK3 and BMAL1.

SEQ ID NO:51: Partial oligopeptide of BMAL1 showing a high score in the local alignment between JNK3 and BMAL1.

SEQ ID NO:52: Partial oligopeptide of JNK3 showing a high score in the local alignment between JNK3 and BMAL1.

SEQ ID NO:53: Partial oligopeptide of BMAL1 showing a high score in the local alignment between JNK3 and BMAL1.

SEQ ID NO:54: Partial oligopeptide of JNK3 showing a high score in the local alignment between JNK3 and BMAL1.

SEQ ID NO:55: Partial oligopeptide of BMAL1 showing a high score in the local alignment between JNK3 and BMAL1.

SEQ ID NO:56: Partial oligopeptide of JNK3 showing a high score in the local alignment between JNK3 and BMAL1.

SEQ ID NO:57: Partial oligopeptide of BMAL1 showing a high score in the local alignment between JNK3 and BMAL1.

SEQ ID NO:58: Partial oligopeptide of JNK3 showing a high score in the local alignment between JNK3 and BMAL1.

SEQ ID NO:59: Partial oligopeptide of BMAL1 showing a high score in the local alignment between JNK3 and BMAL1.

SEQ ID NO:60: Partial oligopeptide of JNK3 showing a high score in the local alignment between JNK3 and BMAL1.

SEQ ID NO:61: Partial oligopeptide of BMAL1 showing a high score in the local alignment between JNK3 and BMAL1.

SEQ ID NO:62: Partial oligopeptide of JNK3 showing a high score in the local alignment between JNK3 and BMAL1.

SEQ ID NO:63: Partial oligopeptide of BMAL1 showing a high score in the local alignment between JNK3 and BMAL1.

SEQ ID NO:64: Partial oligopeptide of JNK3 showing a high score in the local alignment between JNK3 and BMAL2.

SEQ ID NO:65: Partial oligopeptide of BMAL2 showing a high score in the local alignment between JNK3 and BMAL2.

SEQ ID NO:66: Partial oligopeptide of JNK3 showing a high score in the local alignment between JNK3 and BMAL2.

SEQ ID NO:67: Partial oligopeptide of BMAL2 showing a high score in the local alignment between JNK3 and BMAL2.

SEQ ID NO:68: Partial oligopeptide of JNK3 showing a high score in the local alignment between JNK3 and BMAL2.

SEQ ID NO:69: Partial oligopeptide of BMAL2 showing a high score in the local alignment between JNK3 and BMAL2.

SEQ ID NO:70: Partial oligopeptide of JNK3 showing a high score in the local alignment between JNK3 and BMAL2.

SEQ ID NO:71: Partial oligopeptide of BMAL2 showing a high score in the local alignment between JNK3 and BMAL2.

SEQ ID NO:72: Partial oligopeptide of JNK3 showing a high score in the local alignment between JNK3 and BPL1.

SEQ ID NO:73: Partial oligopeptide of BPL1 showing a high score in the local alignment between JNK3 and BPL1.

SEQ ID NO:74: Partial oligopeptide of JNK3 showing a high score in the local alignment between JNK3 and BPL1.

SEQ ID NO:75: Partial oligopeptide of BPL1 showing a high score in the local alignment between JNK3 and BPL1.

SEQ ID NO:76: Partial oligopeptide of JNK3 showing a high score in the local alignment between JNK3 and BPL1

SEQ ID NO:77: Partial oligopeptide of BPL1 showing a high score in the local alignment between JNK3 and BPL1.

SEQ ID NO:78: Partial oligopeptide of JNK3 showing a high score in the local alignment between JNK3 and BPL1.

SEQ ID NO:79: Partial oligopeptide of BPL1 showing a high score in the local alignment between JNK3 and BPL1.

SEQ ID NO:80: Partial sequence which is common in sequence between JNK3 and BPL1.

SEQ ID NO:81: Partial oligopeptide of JNK3 showing a high score in the local alignment between JNK3 and BPL1.

SEQ ID NO:82: Partial oligopeptide of BPL1 showing a high score in the local alignment between JNK3 and BPL1.

SEQ ID NO:83: Partial sequence which is common in sequence between JNK3 and BPL1.

SEQ ID NO:84: Partial oligopeptide of JNK3 showing a high score in the local alignment between JNK3 and BPL1.

SEQ ID NO:85: Partial oligopeptide of BPL1 showing a high score in the local alignment between JNK3 and BPL1.

SEQ ID NO:86: Partial sequence which is common in sequence between JNK3 and BPL1.

SEQ ID NO:87: Partial oligopeptide of JNK3 showing a high score in the local alignment between JNK3 and BPL1.

SEQ ID NO:88: Partial oligopeptide of BPL1 showing a high score in the local alignment between JNK3 and BPL1.

SEQ ID NO:89: Partial oligopeptide of JNK3 showing a high score in the local alignment between JNK3 and BPL1.

SEQ ID NO:90: Partial oligopeptide of BPL1 showing a high score in the local alignment between JNK3 and BPL1.

1. An agent for inhibiting phosphorylation of c-Jun, wherein the agent comprises one or more peptides selected from the following peptide group having a function for interacting with c-Jun N-terminal kinase 3 as active ingredients:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprised of the amino acid sequence represented by SEQ ID NO: 3 in Sequence List,
(vi) a peptide comprising at least one peptide of said (i) to (v), wherein the peptide has a function for inhibiting the phosphorylation of c-Jun by JNK3,
(vii) a peptide comprised of at least 5 consecutive amino acid residues in an amino acid sequence of at least one peptide of said (i) to (v), wherein the peptide has a function for inhibiting the phosphorylation of c-Jun by JNK3,
(viii) a peptide having mutations of one to several amino acid residues in an amino acid sequence of at least one peptide of said (i) to (v), wherein the peptide has a function for inhibiting the phosphorylation of c-Jun by JNK3,
(ix) a peptide having a homology of 70% or more to at least one peptide of said (i) to (v), wherein the peptide has a function for inhibiting the phosphorylation of c-Jun by JNK3,
(x) a peptide comprised of the amino acid sequence represented by SEQ ID NO: 4 in Sequence List, or
(xi) a peptide comprised of the amino acid sequence represented by SEQ ID NO: 5 in Sequence List.

2. The agent for inhibiting phosphorylation of c-Jun according to claim 1, wherein the peptide group consists of (i), (ii), (iii), (iv), (x), and (xi).

3. A method for inhibiting the phosphorylation of c-Jun, wherein the method comprises putting one or more peptides selected from the following peptide group having a function for interacting with c-Jun N-terminal kinase 3 (JNK3) in coexistence with JNK3:
(i) BMAL1,
(ii) BPL1,
(iii) a peptide comprised of the amino acid sequence represented by SEQ ID NO:1 in Sequence List,
(iv) a peptide comprised of the amino acid sequence represented by SEQ ID NO: 2 in Sequence List,
(v) a peptide comprised of the amino acid sequence represented by SEQ ID NO: 3 in Sequence List,
(vi) a peptide comprising at least one peptide of said (i) to (v), wherein the peptide has a function for inhibiting the phosphorylation of c-Jun by JNK3,
(vii) a peptide comprised of at least 5 consecutive amino acid residues in an amino acid sequence of at least one peptide of said (i) to (v), wherein the peptide has a function for inhibiting the phosphorylation of c-Jun by JNK3,
(viii) a peptide having mutations of one to several amino acid residues in an amino acid sequence of at least one peptide of said (i) to (v), wherein the peptide has a function for inhibiting the phosphorylation of c-Jun by JNK3,
(ix) a peptide having a homology of 70% or more to at least one peptide of said (i) to (v), wherein the peptide has a function for inhibiting the phosphorylation of c-Jun by JNK3,
(x) a peptide comprised of the amino acid sequence represented by SEQ ID NO: 4 in Sequence List, or
(xi) a peptide comprised of the amino acid sequence represented by SEQ ID NO: 5 in Sequence List

4. The method for inhibiting the phosphorylation of c-Jun according to claim 3, wherein the peptide group consists of (i), (ii), (iii), (iv), (x), or (xi).

5. An agent for inhibiting the ability of c-Jun to activate transcription, wherein the agent comprises one or more peptides selected from the following peptide group having a function for interacting with c-Jun N-terminal kinase 3 as the active ingredients:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprised of the amino acid sequence represented by SEQ ID NO: 3 in Sequence List,
(vi) a peptide comprising at least one peptide of said (i) to (v), wherein the peptide has a function for inhibiting the phosphorylation of c-Jun by JNK3,
(vii) a peptide comprised of at least 5 consecutive amino acid residues in an amino acid sequence of at least one peptide of said (i) to (v), wherein the peptide has a function for inhibiting the phosphorylation of c-Jun by JNK3,
(viii) a peptide having mutations of one to several amino acid residues in an amino acid sequence of at least one peptide of said (i) to (v), wherein the peptide has a function for inhibiting the phosphorylation of c-Jun by JNK3,
(ix) a peptide having a homology of 70% or more to at least one peptide of said (i) to (v), wherein the peptide has a function for inhibiting the phosphorylation of c-Jun by JNK3,
(x) a peptide comprised of the amino acid sequence represented by SEQ ID NO: 4 in Sequence List, or
(xi) a peptide comprised of the amino acid sequence represented by SEQ ID NO: 5 in Sequence List.

6. A method for inhibiting the ability of c-Jun to activate transcription, wherein the method comprises putting one or more peptides selected from the following peptide group having a function for interacting with c-Jun N-terminal kinase 3 in coexistence with JNK3:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprised of the amino acid sequence represented by SEQ ID NO: 3 in Sequence List,
(vi) a peptide comprising at least one peptide of said (i) to (v), wherein the peptide has a function for inhibiting the phosphorylation of c-Jun by JNK3,
(vii) a peptide comprised of at least 5 consecutive amino acid residues in an amino acid sequence of at least one peptide of said (i) to (v), wherein the peptide has a function for inhibiting the phosphorylation of c-Jun by JNK3,
(viii) a peptide having mutations of one to several amino acid residues in an amino acid sequence of at least one peptide of said (i) to (v), wherein the peptide has a function-for inhibiting the phosphorylation of c-Jun by JNK3,
(ix) a peptide having a homology of 70% or more to at least one peptide of said (i) to (v), wherein the peptide has a function for inhibiting the phosphorylation of c-Jun by JNK3,
(x) a peptide comprised of the amino acid sequence represented by SEQ ID NO: 4 in Sequence List, or
(xi) a peptide comprised of the amino acid sequence represented by SEQ ID NO: 5 in Sequence List.

7. A pharmaceutical composition comprising an effective dose of the agent for inhibiting the phosphorylation of c-Jun according to claim 1.

8. The pharmaceutical composition according to claim 7, wherein the pharmaceutical composition is an agent for preventing and/or treating disease(s) caused by the phosphorylation of c-Jun by c-Jun N-terminal kinase 3.

9. A pharmaceutical composition comprising an effective dose of one or more peptides selected from the following peptide group having a function for interacting with c-Jun N-terminal kinase 3, wherein the pharmaceutical composition is an agent for preventing and/or treating disease(s) caused by the phosphorylation of c-Jun by JNK3:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprised of the amino acid sequence represented by SEQ ID NO: 3 in Sequence List,
(vi) a peptide comprising at least one peptide of said (i) to (v), wherein the peptide has a function for inhibiting the phosphorylation of c-Jun by JNK3,
(vii) a peptide comprised of at least 5 consecutive amino acid residues in an amino acid sequence of at least one peptide of said (i) to (v), wherein the peptide has a function for inhibiting the phosphorylation of c-Jun by JNK3,
(viii) a peptide having mutations of one to several amino acid residues in an amino acid sequence of at least one peptide of said (i) to (v), wherein the peptide has a function for inhibiting the phosphorylation of c-Jun by JNK3,
(ix) a peptide having a homology of 70% or more to at least one peptide of said (i) to (v), wherein the peptide has a function for inhibiting the phosphorylation of c-Jun by JNK3,
(x) a peptide comprised of the amino acid sequence represented by SEQ ID NO: 4 in Sequence List, or
(xi) a peptide comprised of the amino acid sequence represented by SEQ ID NO: 5 in Sequence List.

10. A pharmaceutical composition comprising an effective dose of one or more polynucleotides encoding at least one peptide selected from the following peptide group having a function for interacting with c-Jun N-terminal kinase 3 (JNK3), wherein the pharmaceutical composition is an agent for preventing and/or treating disease(s) caused by the phosphorylation of c-Jun by JNK3:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprised of the amino acid sequence represented by SEQ ID NO: 3 in Sequence List,
(vi) a peptide comprising at least one peptide of said (i) to (v), wherein the peptide has a function for inhibiting the phosphorylation of c-Jun by JNK3,
(vii) a peptide comprised of at least S consecutive amino acid residues in an amino acid sequence of at least one peptide of said (i) to (v), wherein the peptide has a function for inhibiting the phosphorylation of c-Jun by JNK3,
(viii) a peptide having mutations of one to several acid residues in an amino acid sequence of at least one peptide of said (i) to (v), wherein the peptide has a function for inhibiting the phosphorylation of c-Jun by JNK3,
(ix) a peptide having a homology of 70% or more to at least one peptide of said (i) to (v), wherein the peptide has a function for inhibiting the phosphorylation of e-Jun by JNK3,
(x) a peptide comprised of the amino acid sequence represented by SEQ ID NO: 4 in Sequence List, or
(xi) a peptide comprised of the amino acid sequence represented by SEQ ID NO: 5 in Sequence List

11. The pharmaceutical composition according to claim 8, wherein the disease caused by the phosphorylation of c-Jun by c-Jun N-terminal kinase 3 is a neurodegenerative disease.

12. The pharmaceutical composition according to claim 11, wherein the neurodegenerative disease is polyglutamine disease, Huntington's disease, spinocerebellar ataxia, bulbospinal muscular atrophy, dentatorubropallidoluysian atrophy, Alzheimer's disease, Down's disease, Parkinson's disease, Lewy body dementia, multiple system atrophy, familial amyotrophic lateral sclerosis, progressive supranuclear palsy, corticobasal degeneration, Pick's disease, familial British dementia, Creutzfeldt-Jakob disease, Gerstmann-Stranssler syndrome, mad cow disease (bovine spongiform encephalopathy) (BSE), or familial dementia accompanying neuroserpin inclusion bodies.

13. A method for preventing and/or treating disease(s) caused by the phosphorylation of c-Jun by JNK3, wherein the method comprises putting one or more peptides selected from the following peptide group having a function for interacting with c-Jun N-terminal kinase 3 (JNK3) in coexistence with JNK3:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprised of the amino acid sequence represented by SEQ ID NO: 3 in Sequence List,
(vi) a peptide comprising at least one peptide of said (i) to (v), wherein the peptide has a function for inhibiting the phosphorylation of c-Jun by JNK3,
(vii) a peptide comprised of at least 5 consecutive amino acid residues in an amino acid sequence of at least one peptide of said (i) to (v), wherein the peptide has a function for inhibiting the phosphorylation of c-Jun by JNK3,
(viii) a peptide having mutations of one to several acid residues in an amino acid sequence of at least one peptide of said (i) to (v), wherein the peptide has a function for inhibiting the phosphorylation of c-Jun by JNK3,
(ix) a peptide having a homology of 70% or more to at least one peptide of said (i) to (v), wherein the peptide has a function for inhibiting the phosphorylation of c-Jun by JNK3,
(x) a peptide comprised of the amino acid sequence represented by SEQ ID NO: 4 in Sequence List, or
(xi) a peptide comprised of the amino acid sequence represented by SEQ ID NO: 5 in Sequence List.

14. A method for preventing and/or treating disease(s) caused by the phosphorylation of c-Jun by JNK3, wherein the method comprises utilizing one or more polynucleotides encoding at least one peptide selected from the following peptide group having a function for interacting with c-Jun N-terminal kinase 3 (JNK3) to express the peptide encoded by the polynucleotides, wherein the peptide inhibits the phosphorylation of c-Jun by JNK3:
(i)BMAL1,
(ii) BPL1,
(iii) a peptide comprised of the amino acid sequence represented by SEQ ID NO:1 in Sequence List,
(iv) a peptide comprised of the amino acid sequence represented by SEQ ID NO: 2 in Sequence List,
(v) a peptide comprised of the amino acid sequence represented by SEQ ID NO: 3 in Sequence List,
(vi) a peptide comprising at least one peptide of said (i) to (v), wherein the peptide has a function for inhibiting the phosphorylation of c-Jun by JNK3,
(vii) a peptide comprised of at least 5 consecutive amino acid residues in an amino acid sequence of at least one peptide of said (i) to (v), wherein the peptide has a function for inhibiting the phosphoxylation of c-Jun by JNK3,
(viii) a peptide having mutations of one to several amino acid residues in an amino acid sequence of at least one peptide of said (i) to (v), wherein the peptide has a function for inhibiting the phosphorylation of c-Jun by JNK3,
(ix) a peptide having a homology of 70% or more to at least one peptide of said (i) to (v), wherein the peptide has a function for inhibiting the phosphorylation of c-Jun by JNK3,
(x) a peptide comprised of the amino acid sequence represented by SEQ ID NO: 4 in Sequence List, or
(xi) a peptide comprised of the amino acid sequence represented by SEQ ID NO: 5 in Sequence List.

15. A method for preventing and/or treating disease(s) caused by the phosphorylation of c-Jun by JNK3, wherein the method comprises using the pharmaceutical composition according to claim 7.

16. The method for preventing and/or treating disease(s) according to claim 13, wherein the disease caused by the phosphorylation of c-Jun by JNK3 is a neurodegenerative disease.

17. The method for preventing and/or treating disease(s) according to claim 16, wherein the neurodegenerative disease is polyglutamine disease, Huntington's disease, spinocerebellar ataxia, bulbospinal muscular atrophy, dentatorubropallidoluysian atrophy, Alzheimer's disease, Down's disease, Parkinson's disease, Lewy body dementia, multiple system atrophy, familial amyotrophic lateral sclerosis, progressive supranuclear palsy, corticobasal degeneration, Pick's disease, familial British dementia, Creutzfeldt-Jakob disease, Gerstmann-Stranssler syndrome, mad cow disease (bovine spongiform encephalopathy) (BSE), or familial dementia accompanying neuroserpin inclusion bodies.

18. At least one peptide selected from the following peptide group:
(i) a peptide comprised of the amino acid sequence represented by SEQ ID NO: 1 in Sequence List,
(ii) a peptide comprising the peptide comprised of the amino acid sequence represented by SEQ ID NO: 1 in Sequence List,
(iii) a peptide comprised of at least 5 consecutive amino acid residues in the amino acid sequence represented by SEQ ID NO: 1 in Sequence List, or
(iv) a peptide having mutations of one to several amino acids in at least one peptide of (i) to (iii).

19. The peptide according to claim 18, wherein the peptide has a function for interacting with c-Jun. N-terminal kinase 3 (JNK3).

20. A polynucleotide comprising a nucleotide sequence encoding a peptide according to claim 18, or a complementary sequence thereof

21. A polynucleotide comprised of a nucleotide represented by SEQ ID NO: 7 in Sequence List.

22. A polynucleotide which hybridizes with a polynucleotide according to claim 20 under a stringent conditions.

23. A recombinant vector comprising a polynucleotide according to claim 20.

24. The recombinant vector according to claim 23, wherein the recombinant vector is an expression recombinant vector.

25. A transformant transfected with a recombinant vector according to claim 23.

26. A method for producing a peptide according to claim 18, comprising a process for culturing a transformant transfected with an expression recombinant vector comprising a polynucleotide comprising a nucleotide sequence encoding said peptide.

27. An antibody which immunologically recognizes a peptide according to claim 18.

28. A method of identifying a compound that mediates or inhibits the interaction of a peptide according to claim 19 with c-Jun N-terminal kinase 3, wherein the method comprises using at least one selected from the peptide, a polynucleotide encoding the peptide, a recombinant vector comprising the polynucleotide, a transformant transfected with the recombinant vector, or an antibody which immunologically recognizes the peptide.

29. A method of identifying a compound that mediates or inhibits the expression of a polynucleotide encoding a peptide according to claim 19, wherein the method comprises using at least one selected from the polynucleotide encoding the peptide, a recombinant vector comprising the polynucleotide, a transformant transfected with the recombinant vector, or an antibody which immunologically recognizes the peptide.

30. A pharmaceutical composition comprising an effective dose of at least one selected from a peptide according to claim 19, a polynucleotide encoding the peptide, a recombinant vector comprising the polynucleotide, a transformant transfected with the recombinant vector, or an antibody which immunologically recognizes the peptide.

31. An agent for inhibiting the phosphorylation of c-Jun, wherein the agent comprises one or more peptides selected from the following peptide group having a function for interacting with c-Jun N-terminal kinase 3 as the active ingredients:
(i) BMAL1,
(ii) BPL1,
(iii) a peptide comprised of the amino acid sequence represented by SEQ ID NO:1 in Sequence List,
(iv) a peptide comprised of the amino acid sequence represented by SEQ ID NO: 2 in Sequence List,
(v) a peptide comprised of the amino acid sequence represented by SEQ ID NO: 3 in Sequence List, wherein the peptide is human originated,
(vi) a peptide comprising at least one peptide of said (i) to (iv), wherein the peptide has a function for inhibiting the phosphorylation of c-Jun by JNK3,
(vii) a peptide comprising the peptide of said (v), wherein the peptide is human originated and has a function for inhibiting the phosphorylation of c-Jun by JNK3,
(viii) a peptide comprised of at least 5 consecutive amino acid residues in an amino acid sequence of at least one peptide of said (i) to (iv), wherein the peptide has a function for inhibiting the phosphorylation of c-Jun by JNK3,
(ix) a peptide comprised of at least 5 consecutive amino acid residues in an amino acid sequence of the peptide of said (v), wherein the peptide is human originated and has a function for inhibiting the phosphorylation of c-Jun by JNK3,
(x) a peptide having mutations of one to several amino acid residues in an amino acid sequence of at least one peptide of said (i) to (iv), wherein the peptide has a function for inhibiting the phosphorylation of c-Jun by JNK3,
(xi) a peptide having mutations of one to several amino acid residues in an amino acid sequence of the peptide of said (v), wherein the peptide is human originated and has a function for inhibiting the phosphorylation of c-Jun by JNK3,
(xii) a peptide having a homology of 70% or more to at least one peptide of said (i) to (iv), wherein the peptide has a function for inhibiting the phosphorylation of c-Jun by JNK3,
(xiii) a peptide having a homology of 70%/o or more to peptide of said (v), wherein the peptide is human originated and has a function for inhibiting the phosphorylation of c-Jun by JNK3,
(xiv) a peptide comprised of the amino acid sequence represented by SEQ ID NO: 4 in Sequence List, or
(xv) a peptide comprised of the amino acid sequence represented by SEQ ID NO: 5 in Sequence List.

32. An agent for inhibiting the ability to activate transcription of c-Jun, wherein the agent comprises one or more peptides selected from the following peptide group having a function for interacting with c-Jun N-terminal kinase 3 as the active ingredients:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprised of the amino acid sequence represented by SEQ ID NO: 3 in Sequence List and human originated,
(vi) a peptide comprising at least one peptide of said (i) to (iv), wherein the peptide has a function for inhibiting the phosphorylation of c-Jun by JNK3,
(vii) a peptide comprising the peptide of said (v), wherein the peptide is human originated and has a function for inhibiting the phosphorylation of c-Jun by JNK3,
(viii) a peptide comprised of at least 5 consecutive amino acid residues in an amino acid sequence of at least one peptide of said (i) to (iv), wherein the peptide has a function for inhibiting the phosphorylation of c-Jun by JNK3,
(ix) a peptide comprised of at least 5 consecutive amino acid residues in an amino acid sequence of the peptide of said (v), wherein the peptide is human originated and has a function for inhibiting the phosphorylation of c-Jun by JNK3,
(x) a peptide having mutations of one to several amino acid residues in an amino acid sequence of at least one peptide of said (i) to (iv), wherein the peptide has a function for inhibiting the phosphorylation of c-Jun by JNK3,
(xi) a peptide having mutations of one to several amino acid residues in an amino acid sequence of the peptide of said (v), wherein the peptide is human originated and has a function for inhibiting the phosphorylation of c-Jun by JNK3,
(xii) a peptide having a homology of 70% or more to at least one peptide of said (i) to (iv), wherein the peptide has a function for inhibiting the phosphorylation of c-Jun by JNK3,
(xiii) a peptide having a homology of 70% or more to peptide of said (v), wherein the peptide is human originated and has a function for inhibiting the phosphorylation of c-Jun by JNK3,
(xiv) a peptide comprised of the amino acid sequence represented by SEQ ID NO: 4 in Sequence List, or
(xv) a peptide comprised of the amino acid sequence represented by SEQ ID NO: 5 in Sequence List.

33. A pharmaceutical composition comprising an effective dose-of an agent for inhibiting the ability to activate transcription of c-Jun according to claim 32.

34. The pharmaceutical composition according to claim 33, wherein the pharmaceutical composition is an agent for preventing and/or treating disease(s) caused by the phosphorylation of c-Jun by c-Jun N-terminal kinase 3.

35. A pharmaceutical composition comprising an effective dose of one or more peptides selected from the following peptide group having a function for interacting with c-Jun N-terminal kinase 3 (JNK3), wherein the pharmaceutical composition is an agent for preventing and/or treating disease(s) caused by the phosphorylation of c-Jun by JNK3:
(i) BMAL1,
(ii) BPL1,
(iii) a peptide comprised of the amino acid sequence represented by SEQ. ID NO: 1 in Sequence List,
(iv) a peptide comprised of the amino acid sequence represented by SEQ ID NO: 2 in Sequence List,
(v) a peptide comprised of the amino acid sequence represented by SEQ ID NO: 3 in Sequence List and human originated,
(vi) a peptide comprising at least one peptide of said (i) to (iv), wherein the peptide has a function for inhibiting the phosphorylation of c-Jun by JNK3,
(vii) a peptide comprising the peptide of said (v), wherein the peptide is human originated and has a function for inhibiting the phosphorylation of c-Jun by JNK3,
(viii) a peptide comprised of at least 5 consecutive amino acid residues in an amino acid sequence of at least one peptide of said (i) to (iv), wherein the peptide has a function for inhibiting the phosphorylation of c-Jun by JNK3,
(ix) a peptide comprised of at least 5 consecutive amino acid residues in an amino acid sequence of the peptide of said (v), wherein the peptide is human originated and has a function for inhibiting the phosphorylation of c-Jun by JNK3,
(x) a peptide having mutations of one to several amino acid residues in an amino acid sequence of at least one peptide of said (i) to (iv), wherein the peptide has a function for inhibiting the phosphorylation of c-Jun by JNK3,
(xi) a peptide having mutations of one to several amino acid residues in an amino acid sequence of the peptide of said (v), wherein the peptide is human originated and has a function for inhibiting the phosphorylation of c-Jun by JNK3,
(xii) a peptide having a homology of 70% or more to at least one peptide of said (i) to (iv), wherein the peptide has a function for inhibiting the phosphorylation of c-Jun by JNK3,
(xiii) a peptide having a homology of 70% or more to peptide of said (v), wherein the peptide is human originated and has a function for inhibiting the phosphorylation of c-Jun by JNK3,
(xiv) a peptide comprised of the amino acid sequence represented by SEQ ID NO: 4 in Sequence List, or
(xv) a peptide comprised of the amino acid sequence represented by SEQ ID NO: 5 in Sequence List.

36. A pharmaceutical composition comprising an effective dose of one or more polynucleotides encoding at least one peptide selected from the following peptide group having a function for interacting with c-Jun N-terminal kinase (JNK3), wherein the pharmaceutical composition is an agent for preventing and/or treating disease(s) caused by the phosphorylation of c-Jun caused by JNK3:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprised of the amino acid sequence represented by SEQ ID NO: 3 in Sequence List and human originated,
(vi) a peptide comprising at least one peptide of said (i) to (iv), wherein the peptide has a function for inhibiting the phosphorylation of c-Jun by JNK3,
(vii) a peptide comprising the peptide of said (v), wherein the peptide is human originated and has a function for inhibiting the phosphorylation of c-Jun by JNK3,
(viii) a peptide comprised of at least 5 consecutive amino acid residues in an amino acid sequence of at least one peptide of said (i) to (iv), wherein the peptide has a function for inhibiting the phosphorylation of c-Jun by JNK3,
(ix) a peptide comprised of at least 5 consecutive amino acid residues in an amino acid sequence of the peptide of said (v), wherein the peptide is human originated and has a function for inhibiting the phosphorylation of c-Jun by JNK3,
(x) a peptide having mutations of one to several amino acid residues in an amino acid sequence of at least one peptide of said (i) to (iv), wherein the peptide has a function for inhibiting the phosphorylation of c-Jun by JNK3,
(xi) a peptide having mutations of one to several amino acid residues in an amino acid sequence of the peptide of said (v), wherein the peptide is human originated and has a function for inhibiting the phosphorylation of c-Jun by JNK3,
(xii) a peptide having a homology of 70% or more to at least one peptide of said (i) to (iv), wherein the peptide has a function for inhibiting the phosphorylation of c-Jun by JNK3,
(xiii) a peptide having a homology of 70% or more to peptide of said (v), wherein the peptide is human originated and has a function for inhibiting the phosphorylation of c-Jun by JNK3,
(xiv) a peptide comprised of the amino acid sequence represented by SEQ ID NO: 4 in Sequence List, or
(xv) a peptide comprised of the amino acid sequence represented by SEQ ID NO: 5 in Sequence List.

37. The pharmaceutical composition according to at least one of claim 34, wherein the disease caused by the phosphorylation of c-Jun by c-Jun N-terminal kinase 3 is a neurodegenerative disease.

38. The pharmaceutical composition according to claim 37, wherein the neurodegenerative disease is polyglutamine disease, Huntington's disease, spinocerebellar ataxia, bulbospinal muscular atrophy, dentatorubropallidoluysian atrophy, Alzheimer's disease, Down's disease, Parkinson's disease, Lewy body dementia, multiple system atrophy; familial amyotrophic lateral sclerosis, progressive supranuclear palsy, corticobasal degeneration, Pick's disease, familial British dementia, Creutzfeldt-Jakob disease, Gerstmann-Stranssler syndrome, mad cow disease (bovine spongiform encephalopathy) (BSE), or familial dementia accompanying neuroserpin inclusion bodies.

39. An agent for inhibiting phosphorylation of c-Jun, wherein the agent comprises one or more peptides selected from the following peptide group having a function for interacting with c-Jun N-terminal kinase 3 as the active ingredients:
(i) BMAL1,
(ii) BPL 1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprising at least one peptide of said (i) to (iv), wherein the peptide has a function for inhibiting the phosphorylation of c-Jun by JNK3,
(vi) a peptide comprised of at least 5 consecutive amino acid residues in an amino acid sequence of at least one peptide of said (1) to (iv), wherein the peptide has a function for inhibiting the phosphorylation of c-Jun by JNK3,
(vii) a peptide having mutations of one to several amino acid residues in an amino acid sequence of at least one peptide of said (i) to (iv), wherein the peptide has a function for inhibiting the phosphorylation of c-Jun by JNK3,
(viii) a peptide having a homology of 70% or more to at least one peptide of said (i) to (iv), wherein the peptide has a function for inhibiting the phosphorylation of c-Jun by JNK3,
(ix) a peptide comprised of the amino acid sequence represented by SEQ ID NO: 4 in Sequence List, or.
(x) a peptide comprised of the amino acid sequence represented by SEQ ID NO: 5 in Sequence List.

40. A method for inhibiting the phosphorylation of c-Jun, wherein the method comprises putting one or more peptides selected from the following peptide group having a function for interacting with c-Jun N-terminal kinase 3 (JNK3) in coexistence with JNK3:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprising at least one of said (i) to (iv), wherein the peptide has a function for inhibiting the phosphorylation of c-Jun by JNK3,
(vi) a peptide comprised of at least 5 consecutive amino acid residues in an amino acid sequence of at least one peptide of said (i) to (iv), wherein the peptide has a function for inhibiting the phosphorylation of c-Jun by JNK3,
(vii) a peptide having mutations of one to several amino acid residues in an amino acid sequence of at least one peptide of said (i) to (iv), wherein the peptide has a function for inhibiting the phosphorylation of c-Jun by JNK3,
(viii) a peptide having a homology of 70% or more to at least one peptide of said (i) to (iv), wherein the peptide has a function for inhibiting the phosphorylation of c-Jun by JNK3,
(ix) a peptide comprised of the amino acid sequence represented by SEQ ID NO: 4 in Sequence List, or
(x) a peptide comprised of the amino acid sequence represented by SEQ ID NO: 5 in Sequence List.

41. The method for inhibiting the phosphorylation of c-Jun according to claim 40, wherein the peptide group consists of (i), (ii), (iii), (iv), (ix), or (x).

42. An agent for inhibiting the ability of c-Jun to activate transcription, wherein the agent comprises one or more than two peptides selected from the following peptide group having a function for interacting with c-Jun N-terminal kinase 3 as the active ingredients:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprising at least one peptide of said (i) to (iv), wherein the peptide has a function for inhibiting the phosphorylation of c-Jun by JNK3,
(vi) a peptide comprised of at least 5 consecutive amino acid residues in an amino acid sequence of at least one peptide of said (i) to (iv), wherein the peptide has a function for inhibiting the phosphorylation of c-Jun by JNK3,
(vii) a peptide having mutations of one to several amino acid residues in an amino acid sequence of at least one peptide of said (i) to (iv), wherein the peptide has a function for inhibiting the phosphorylation of c-Jun by JNK3,
(viii) a peptide having a homology of 70% or more to at least one peptide of said (i) to (iv), wherein the peptide has a function for inhibiting the phosphorylation of c-Jun by JNK3,
(ix) a peptide comprised of the amino acid sequence represented by SEQ ID NO: 4 in Sequence List, or
(x) a peptide comprised of the amino acid sequence represented by SEQ ID NO: 5 in Sequence List.

43. A method for inhibiting the ability of c-Jun to activate transcription, wherein the method comprises putting one or more peptides selected from the following peptide group having a function for interacting with c-Jun N-terminal kinase 3 (INK) in coexistence with JNK3:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprising at least one peptide of said (i) to (iv), wherein the peptide has a function for inhibiting the phosphorylation of c-Jun by JNK3,
(vi) a peptide comprised of at least 5 consecutive amino acid residues in an amino acid sequence of at least one peptide of said (i) to (iv), wherein the peptide has a function for inhibiting the phosphorylation of c-Jun by JNK3,
(vii) a peptide having mutations of one to several amino acid residues in an amino acid sequence of at least one peptide of said (i) to (iv), wherein the peptide has a function for inhibiting the phosphorylation of c-Jun by JNK3,
(viii) a peptide having a homology of 70% or more to at least one peptide of said (i) to (iv), wherein the peptide has a function for inhibiting the phosphorylation of c-Jun by JNK3,
(ix) a peptide comprised of the amino acid sequence represented by SEQ ID NO: 4 in Sequence List, or
(x) a peptide comprised of the amino acid sequence represented by SEQ ID NO: 5 in Sequence List.

44. A pharmaceutical composition comprising an effective dose of an agent for inhibiting phosphorylation of c-Jun according to claim 39, or an agent for inhibiting the ability of c-Jun to activate transcription.

45. A pharmaceutical composition according to claim 44, wherein the pharmaceutical composition is an agent for preventing and/or treating disease(s) caused by the phosphorylation of c-Jun by JNK3.

46. A pharmaceutical composition comprising an effective dose of one or more peptides selected from the following peptide group having a function for interacting with c-Jun N-terminal kinase (JNK3), wherein the pharmaceutical composition is an agent for preventing and/or treating disease(s) caused by the phosphorylation of c-Jun by JNK3:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprising at least one peptide of said (i) to (iv), wherein the peptide has a function for inhibiting the phosphorylation of c-Jun by JNK3,
(vi) a peptide comprised of at least 5 consecutive amino acid residues in an amino acid sequence of at least one peptide of said (i) to (iv), wherein the peptide has a function for inhibiting the phosphorylation of c-Jun by JNK3,
(vii) a peptide having mutations of one to several amino acid residues in an amino acid sequence of at least one peptide of said (i) to (iv), wherein the peptide has a function for inhibiting the phosphorylation of c-Jun by JNK3,
(viii) a peptide having a homology of 70% or more to at least one peptide of said (i) to (iv), wherein the peptide has a function for inhibiting the phosphorylation of c-Jun by JNK3,
(ix) a peptide comprised of the amino acid sequence represented by SEQ ID NO: 4 in Sequence List, or
(x) a peptide comprised of the amino acid sequence represented by SEQ ID NO: 5 in Sequence List.

47. A pharmaceutical composition comprising an effective dose of one or more polynucleotides encoding at least one peptide selected from the following peptide group having a function for interacting with c-Jun N-terminal kinase (JNK3), wherein the pharmaceutical composition is an agent for preventing and/or treating disease(s) caused by the phosphorylation of c-Jun by JNK3:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprising at least one peptide of said (i) to (iv), wherein the peptide has a function for inhibiting the phosphorylation of c-Jun by JNK3,
(vi) a peptide comprised of at least 5 consecutive amino acid residues in an amino acid sequence of at least one peptide of said (i) to (iv), wherein the peptide has a function for inhibiting the phosphorylation of c-Jun by JNK3,
(vii) a peptide having mutations of one to several amino acid residues in an amino acid sequence of at least one peptide of said (i) to (iv), wherein the peptide has a function for inhibiting the phosphorylation of c-Jun by JNK3,
(viii) a peptide having a homology of 70% or more to at least one peptide of said (i) to (iv), wherein the peptide has a function for inhibiting the phosphorylation of c-Jun by JNK3,
(ix) a peptide comprised of the amino acid sequence represented by SEQ ID NO: 4 in Sequence List, or
(x) a peptide comprised of the amino acid sequence represented by SEQ ID NO: 5 in Sequence List.

48. The pharmaceutical composition according to claim 45, wherein the disease caused by the phosphorylation of c-Jun by c-Jun N-terminal kinase is a neurodegenerative disease.

49. The pharmaceutical composition according to claim 48, wherein the neurodegenerative disease is polyglutamine disease, Huntington's disease, spinocerebellar ataxia, bulbospinal muscular atrophy, dentatorubropailidoluysian atrophy, Alzheimer's disease, Down's disease, Parkinson's disease, Lewy body dementia, multiple system atrophy, familial amyotrophic lateral sclerosis, progressive supranuclear palsy, corticobasal degeneration, Pick's disease, familial British dementia, Creutzfeldt-Jakob disease, Gerstnaum-Stranssler syndrome, mad cow disease (bovine spongiform encephalopathy) (BSE), or familial dementia accompanying neuroserpin inclusion bodies.

50. A method for preventing and/or treating disease(s) caused by the phosphorylation of c-Jun by JNK3, wherein the method comprises putting one or more peptides selected from the following peptide group having a function for interacting with c-Jun N-terminal kinase (JNK3) in coexistence with JNK3:
(i) BMAL1,
(ii) BPL1,
(iii) a peptide comprised of the amino acid sequence represented by SEQ ID NO: 1 in Sequence List,
(iv) a peptide comprised of the amino acid sequence represented by SEQ ID NO: 2 in Sequence List,
(v) a peptide comprising at least one peptide of said (i) to (iv), wherein the peptide has a function for inhibiting the phosphorylation of c-Jun by JNK3,
(vi) a peptide comprised of at least 5 consecutive amino acid residues in an amino acid sequence of at least one peptide of said (i) to (iv), wherein the peptide has a function for inhibiting the phosphorylation of c-Jun by JNK3,
(vii) a peptide having mutations of one to several amino acid residues in an amino acid sequence of at least one peptide of said (i) to (iv), wherein the peptide has a function for inhibiting the phosphorylation of e-Jun by JNK3,
(viii) a peptide having a homology of 70% or more to at least one peptide of said (i) to (iv), wherein the peptide has a function for inhibiting the phosphorylation of c-Jun by JNK3,
(ix) a peptide comprised of the amino acid sequence represented by SEQ ID NO: 4 in Sequence List, or
(x) a peptide comprised of the amino acid sequence represented by SEQ ID NO: 5 in Sequence List.

51. A method for preventing and/or treating disease(s) caused by the phosphorylation of c-Jun by JNK3, wherein the method comprises utilizing one or more polynucleotides encoding at least one peptide selected from the following peptide group having a function for interacting with c-Jun N-terminal kinase (JNK3) to express the peptide encoded by the polynucleotides, wherein the peptide inhibits the phosphorylation of c-Jun by JNK3:
(i) BMAL1,
(ii) BPL1,
(iii) a peptide composed of the amino acid sequence represented by SEQ ID NO: 1 in Sequence List,
(iv) a peptide composed of the amino acid sequence represented by SEQ ID NO: 2 in Sequence List,
(v) a peptide comprising at least one peptide of said (i) to (iv), wherein the peptide has a function for inhibiting the phosphorylation of c-Jun by JNK3,
(vi) a peptide composed of at least 5 consecutive amino acid residues in an amino acid sequence of at least one peptide of said (i) to (iv), wherein the peptide has a function for inhibiting the phosphorylation of c-Jun by JNK3,
(vii) a peptide having mutations of one to several amino acid residues in an amino acid sequence of at least one peptide of said (i) to (iv), wherein the peptide has a function for inhibiting the phosphorylation of c-Jun by JNK3,
(viii) a peptide having a homology of 70% or more to at least one peptide of said (i) to (iv), wherein the peptide has a function for inhibiting the phosphorylation of c-Jun by JNK3,
(ix) a peptide comprised of the amino acid sequence represented by SEQ ID NO: 4 in Sequence List, or
(x) a peptide comprised of the amino acid sequence represented by SEQ ID NO: 5 in Sequence List.

52. A method for preventing and/or treating disease(s) caused by the phosphorylation of c-Jun by JNK3, wherein the method comprises using a pharmaceutical composition according to claim 44.

53. The method for preventing and/or treating disease(s) according to claim 50, wherein the disease caused by the phosphorylation of c-Jun by c-Jun N-terminal kinase 3 is a neurodegenerative disease.

54. The method for preventing and/or treating disease(s) according to claim 53, wherein the neurodegenerative disease is polyglutamine disease, Huntington's disease, spinocerebellar ataxia, bulbospinal muscular atrophy, dentatorubropallidoluysian atrophy, Alzheimer's disease, Down's disease, Parkinson's disease, Lewy body dementia, multiple system atrophy, familial amyotrophic lateral sclerosis, progressive supranuclear palsy, corticobasal degeneration, Pick's disease, familial British dementia, Creutzfeldt-Jakob disease, Gerstmann-Stranssler syndrome, mad cow disease (bovine spongiform encephalopathy) (BSE), or familial dementia accompanying neuroserpin inclusion bodies.

55. A pharmaceutical composition comprising an effective dose of said agent, according to claim 31 for inhibiting the ability to activate transcription of c-Jun.

56. The pharmaceutical composition according to claim 32, wherein the pharmaceutical composition is an agent for preventing and/or treating disease(s) caused by the phosphorylation of c-Jun by N-terminal kinase 3.


SEQUENCE LISTING (90 sequences)

SEQ ID NO: 1 - protein, 786 aa, Homo sapiens
MAPGTGSSTAVNSCSPQSLSSVLGSGFGELAPPKMANITSSQILDQLKAPSLGQFTTTPSTQQNSTSHPTTTTSWDLKPPTSQSSVLSHLDFKSQPEPSPVLSQLSQRQQHQSQAVTVPPPGLESFPSQAKLRESTPGDSPSTVNKLLQLPSTTIENISVSVHQPQPKHIKLAKRRIPPASKIPASAVEMPGSADVTGLNVQFGALEFGSEPSLSEFGSAPSSENSNQIPISLYSKSLSEPLNTSLSMTSAVQNSTYTTSVITSCSLTSSSLNSASPVAMSSSYDQSSVHNRIPYQSPVSSSESAPGTIMNGHGGGRSQQTLDTPKTTGPPSALPSVSSLPSTTSCTALLPSTSQHTGDLTSSPLSQLSSSLSSHQSSLSAHAALSSSTSHTHASVESASSHQSSATFSTAATSVSSSASSGVSLSSSMNTANSLCLGGTPASASSSSSRAAPLVTSGKAPPNLPQGVPPLLHNQYLVGPGGLLPAYPIYGYDELQMLQSRLPVDYYGIPFAAPTALASRDRSLANNPYPGDVTKFGRGDSASPAPATTPAQPQQSQSQTHHTAQQPFVNPALPPGYSYTGLPYYTGMPSAFQYGPTMFVPPASAKQHGVNLSTPTPPFQQASGYGQHGYSTGYDDLTQGTAAGDYSKGGYAGSSQAPNKSAGSGPGKGVSVSSSTTGLPDMTGSVYNKTQTFDKQGFHAGTPPPFSLPSVLGSTGPLASGAAPGYAPPPFLHILPAHQQPHSQLLHHHLPQDAQSGSGQRSQPSSLQPKSQASKPAYGNSPYWTN

SEQ ID NO: 2 - protein, 579 aa, Homo sapiens
LSQEEGVFAQDLEPAPIEDGIVYPEPSDNPTMDTSEFQVQAPARGTLGRVYPGSRSSEKHSPDSACSVDYSSSCLSSPEHPTEDSESTEPLSVDGISSDLEEPAEGDEEEEEEEGGMGPYGLQEGSPQTPDQEQFLKQHFETLASGAAPGAPVQVPERSESRSISSRFLLQVQTRPLREPSPSSSSLALMSRPAQVPQASGEQPRGNGANPPGAPPEVEPSSGNPSPQQAASVLLPRCRLNPDSSWAPKRVATASPFSGLQKAQSVHSLVPQERHEASLQAPSPGALLSREIEAQDGLGSLPPADGPPSRPHSYQNPTTSSMAKISRSISVGENLGLVAEPQAHAPIRVSPLSKLALPSRAHLVLDIPKPLPDRPTLAAFSPVTKGRAPGEAEKPGFPVGLGKAHSTTERWACLGEGTTPKPRTECQAHPGPSSPCAQQLPVSSLFQGPENLQPPPPEKTPNPMECTKPGAALSQDSAVSLEQCEQLVAELRGSVRQAVRLYHSVAGCKMPSAEQSRIAQLLRDTFSSVRQELEAVAGAVLSSPGSSPGAVGAEQTQALLEQYSELLLRAVERRMERKL

SEQ ID NO: 3 - protein, 1217 aa, Homo sapiens
SNKVSSRVTAVSFSEDCSYFVTAGNRHIKFWYLDDSKTSKVNATVPLLGRSGLLGELRNNLFTDVACGRGKKADSTFCITSSGLLCEFSDRRLLDKWVELRVYPEVKDSNQACLPPSSFITCSSDNTIRLWNTESSGVHGSTLHRNILSSDLIKIIYVDGNTQALLDTELPGGDKADASLLDPRVGIRSVCVSPNGQHLASGDRMGTLRVHELQSLSEMLKVEAHDSEILCLEYSKPDTGLKLLASASRDRLIHVLDAGREYSLQQTLDEHSSSITAVKFAASDGQVRMISCGADKSIYFRTAQKSGDGVQFTRTHHVVRKTTLYDMDVEPSWKYTAIGCQDRNIRIFNISSGKQKKLFKGSQGEDGTLIKVQTDPSGIYIATSCSDKNLSIFDFSSGECVATMFGHSEIVTGMKFSNDCKHLISVSGDSCIFVWRLSSEMTISMRQRLAELRQRQRGGKQQGPSSPQRASGPNRHQAPSMLSPGPALSSDSDKEGEDEGTEEELPALPVLAKSTKKALASVPSPALPRSLSHWEMSRAQESVGFLDPAPAANPGPRRRGRWVQPGVELSVRSMLDLRQLETLAPSLQDPSQDSLAIIPSGPRKHGQEALETSLTSQNEKPPRPQASQPCSYPHIIRLLSQEEGVFAQDLEPAPIEDGIVYPEPSDNPTMDTSEFQVQAPARGTLGRVYPGSRSSEKHSPDSACSVDYSSSCLSSPEHPTEDSESTEPLSVDGISSDLEEPAEGDEEEEEEEGGMGPYGLQEGSPQTPDQEQFLKQHFETLASGAAPGAPVQVPERSESRSISSRFLLQVQTRPLREPSPSSSSLALMSRPAQVPQASGEQPRGNGANPPGAPPEVEPSSGNPSPQQAASVLLPRCRLNPDSSWAPKRVATASPFSGLQKAQSVHSLVPQERHEASLQAPSPGALLSREIEAQDGLGSLPPADGPPSRPHSYQNPTTSSMAKISRSISVGENLGLVAEPQAHAPIRVSPLSKLALPSRAHLVLDIPKPLPDRPTLAAFSPVTKGRAPGEAEKPGFPVGLGKAHSTTERWACLGEGTTPKPRTECQAHPGPSSPCAQQLPVSSLFQGPENLQPPPPEKTPNPMECTKPGAALSQDSAVSLEQCEQLVAELRGSVRQAVRLYHSVAGCKMPSAEQSRIAQLLRDTFSSVRQELEAVAGAVLSSPGSSPGAVGAEQTQALLEQYSELLLRAVERRMERKL

SEQ ID NO: 4 - protein, 626 aa, Homo sapiens
MADQRMDISSTISDFMSPGPTDLLSSSLGTSGVDCNRKRKGSSTDYQESMDTDKDDPHGRLEYTEHQGRIKNAREAHSQIEKRRRDKMNSFIDELASLVPTCNAMSRKLDKLTVLRMAVQHMKTLRGATNPYTEANYKPTFLSDDELKHLILRAADGFLFVVGCDRGKILFVSESVFKILNYSQNDLIGQSLFDYLHPKDIAKVKEQLSSSDTAPRERLIDAKTGLPVKTDITPGPSRLCSGARRSFFCRMKCNRPSVKVEDKDFPSTCSKKKADRKSFCTIHSTGYLKSWPPTKMGLDEDNEPDNEGCNLSCLVAIGRLHSHVVPQPVNGEIRVKSMEYVSRHAIDGKFVFVDQRATAILAYLPQELLGTSCYEYFHQDDIGHLAECHRQVLQTREKITTNCYKFKIKDGSFITLRSRWFSFMNPWTKEVEYIVSTNTVVLANVLEGGDPTFPQLTASPHSMDSMLPSGEGGPKRTHPTVPGIPGGTRAGAGKIGRMIAEEIMEIHRIRGSSPSSCGSSPLNITSTPPPDASSPGGKKILNGGTPDIPSSGLLSGQAQENPGYPYSDSSSILGENPHIGIDMIDNDQGSSSPSNDEAAMAVIMSLLEADAGLGGPVDFSDLPWPL

SEQ ID NO: 5 - protein, 726 aa, Homo sapiens
MEDRLHMDNGLVPQKIVSVHLQDSTLKEVKDQVSNKQAQILEPKPEPSLEIKPEQDGMEHVGRDDPKALGEEPKQRRGSASGSEPAGDSDRGGGPVEHYHLHLSSCHECLELENSTIESVKFASAENIPDLPYDYSSSLESVADETSPEREGRRVNLTGKAPNILLYVGSDSQEALGRFHEVRSVLADCVDIDSYILYHLLEDSALRDPWTDNCLLLVIATRESIPEDLYQKFMAYLSQGGKVLGLSSSFTFGGFQVTSKGALHKTVQNLVFSKADQSEVKLSVLSSGCRYQEGPVRLSPGRLQGHLENEDKDRMIVHVPFGTRGGEAVLCQVHLELPPSSNIVQTPEDFNLLKSSNFRRYEVLREILTTLGLSCDMKQVPALTPLYLLSAAEEIRDPLMQWLGKHVDSEGEIKSGQLSLRFVSSYVSEVEITPSCIPVVTNMEAFSSEHFNLEIYRQNLQTKQLGKVILFAEVTPTTMRLLDGLMFQTPQEMGLIVIAARQTEGKGRGGNVWLSPVGCALSTLLISIPLRSQLGQRIPFVQHLMSVAVVEAVRSIPEYQDINLRVKWPNDIYYSDLMKIGGVLVNSTLMGETFYILIGCGFNVTNSNPTICINDLITEYNKQHKAELKPLRADYLIARVVTVLEKLIKEFQDKGPNSVLPLYYRYWVHSGQQVHLGSAEGPKVSIVGLDDSGFLQVHQEGGEVVTVHPDGNSFDMLRNLILPKRR

SEQ ID NO: 6 - protein, 757 aa, Homo sapiens
LAPPKMANITSSQILDQLKAPSLGQFTTTPSTQQNSTSHPTTTTSWDLKPPTSQSSVLSHLDFKSQPEPSPVLSQLSQRQQHQSQAVTVPPPGLESFPSQAKLRESTPGDSPSTVNKLLQLPSTTIENISVSVHQPQPKHIKLAKRRIPPASKIPASAVEMPGSADVTGLNVQFGALEFGSEPSLSEFGSAPSSENSNQIPISLYSKSLSEPLNTSLSMTSAVQNSTYTTSVITSCSLTSSSLNSASPVAMSSSYDQSSVHNRIPYQSPVSSSESAPGTIMNGHGGGRSQQTLDTPKTTGPPSALPSVSSLPSTTSCTALLPSTSQHTGDLTSSPLSQLSSSLSSHQSSLSAHAALSSSTSHTHASVESASSHQSSATFSTAATSVSSSASSGVSLSSSMNTANSLCLGGTPASASSSSSRAAPLVTSGKAPPNLPQGVPPLLHNQYLVGPGGLLPAYPIYGYDELQMLQSRLPVDYYGIPFAAPTALASRDGSLANNPYPGDVTKFGRGDSASPAPATTPAQPQQSQSQTHHTAQQPFVNPALPPGYSYTGLPYYTGMPSAFQYGPTMFVPPASAKQHGVNLSTPTPPFQQASGYGQHGYSTGYDDLTQGTAAGDYSKGGYAGSSQAPNKSAGSGPGKGVSVSSSTTGLPDMTGSVYNKTQTFDKQGFHAGTPPPFSLPSVLGSTGPLASGAAPGYAPPPFLHILPAHQQPHSQLLHHHLPQDAQSGSGQRSQPSSLQPKSQASKPAYGNSPYWTN

SEQ ID NO: 7 - DNA, 3006 bp, Homo sapiens
atggcaccagggactggcagctccactgccgtcaactcctgttctcctcagagcctgtcatccgtccttggctcaggatttggagagcttgcaccaccaaaaatggcaaacatcaccagctcccagattttggaccagttgaaagctccgagtttgggccagtttaccaccaccccaagtacacagcagaatagtacaagtcaccctacaactactacttcttgggacctcaagcccccaacatcccagtcctcagtcctcagtcatcttgacttcaaatctcaacctgagccatccccagttcttagccagttgagccagcgacaacagcaccagagccaggcagtcactgttcctcctcctggtttggagtcctttccttcccaggcaaaacttcgagaatcaacacctggagacagtccctccactgtgaacaagcttttgcagcttcccagcacgaccattgaaaatatctctgtgtctgtccaccagccacagcccaaacacatcaaacttgctaagcggcggatacccccagcttctaagatcccagcttctgcagtggaaatgcctggttcagcagatgtcacaggattaaatgtgcagtttggggctctggaatttgggtcagaaccttctctctctgaatttggatcagctccaagcagtgaaaatagtaatcagattcccatcagcttgtattcgaagtctttaagtgagcctttgaatacatctttatcaatgaccagtgcagtacagaactccacatatacaacttccgtcattacctcctgcagtctgacaagctcatcactgaattctgctagtccagtagcaatgtcttcctcttatgaccagagttctgtgcataacaggatcccataccaaagccctgtgagttcatcagagtcagctccaggaaccatcatgaatggacatggtggtggtcgaagtcagcagacactagacactccaaagacaacaggccctccctctgccctcccgtctgtgagctccctgcccagcaccacctcctgcactgcacttctgccgtccacatcccagcacactggcgacctgactagcagccctctctctcagcttagcagttcgctctccagccaccagagcagcctctctgcacatgcagccctctcctcgagcacgtcacacacacatgccagtgtggagagcgcctcttcccaccagtcctcagccaccttctccacggcagcgacctccgtctcaagttccgcatcctcaggcgtcagcctgtccagtagcatgaacaccgcgaacagcctctgtctgggtgggacccccgcgagtgcatccagcagcagtagcagggccgcgcccttggtgacctcaggcaaagcacccccaaacttacctcagggggtgcctcccctgctgcacaaccagtacctcgtaggtcccggaggactgcttcctgcctacccgatctatggctatgacgagctccagatgctgcagtcacggctgccagtggactactatggaattccctttgctgcacccacagcgcttgccagccgagatgggagcctagctaataatccatatccaggtgatgtcacaaagtttggccgtggggactctgcatcccctgcacccgctaccacaccagctcagccacagcagagccaatcacagacccaccacacagcccagcagcccttcgtgaatcctgcactgccacctggctatagctacactggtcttccctactacacaggcatgcccagtgccttccagtatggccccaccatgtttgtccctccagcctcagccaagcaacatggggtgaacctcagcactcccacacctcccttccagcaggccagtggttatggccagcacggctacagtacaggttatgacgacctgacccaggggacagcagcaggagactactccaaaggtggctatgctggatcatcgcaggcaccaaacaagtctgcaggttctgggcctggcaaaggagtatcagtgtcttcaagcaccactggtctacctgatatgactggttctgtctacaataagacacagacttttgacaagcagggatttcatgcagggacgcctccacctttcagcctgccctcggtcttgggctccactgggcccctggcctcgggagcggcccctggctatgcacccccaccattcctacacatcttgccagcccaccagcagccccactcacagctgctgcaccaccaccttccgcaggatgcacagagtggctcgggtcagcgcagccagcccagctccctgcagcccaagtctcaagcctccaaacctgcctacggcaactctccatactggacaaactaaacccagaagagaggggtgggctggggcaaggcttatcctgggcaggagagaacacacgagcacgtatttgggagcccagtgccctttcctagaattcccgacatgtgtcagccatgcctctgtggggagtctgcctcccagactggctactgtatgtaatgtatttatgtatgtatttgtaaatgtgatagaagtctgggggggagttgggggatggcggcagatgttagccaggtctgccctccccattcaagccccttctccactgtagcaaaataagcacccccaccccatctgccttcaggtcttcttcacagcctgcactgcccagtgggccactaggggcagtctctggaagggctggttcaaggctgtttgggtataggggtcaggtaccaatgaagaatcacgacttgtctcactcctttggaaattgttttctttcctgtgtaattacttcatacctctgtttttgagaaactgttccgtttgtcatctgtcatggtctccttccaccaaatcttcatctgggaatagcagcggtatccctccacccaagtatggccacctgtttgtcttcatatagaacaggggcttctggtctggtcatgtccctagagacttactagagactggctgaccatg

SEQ ID NO: 8 - DNA, 3655 bp, Homo sapiens
ctccaacaaggtgtccagtcgggtgacagcagtgtccttctctgaggattgcagctactttgtcactgcaggcaaccgacacatcaaattctggtatctcgatgacagcaagacctcaaaggtgaatgccactgtgcccttgctgggccgctcagggctgctgggagagctacggaacaacctattcactgatgtggcctgtggcagaggaaaaaaggcggacagtaccttctgcatcacgtcctcagggctgctgtgcgagttcagtgatcgaaggcttttggacaagtgggtggagctgagggtctaccccgaggtgaaggatagtaaccaggcctgcctgccccccagttcctttattacctgctcctcagacaacaccatccgcctgtggaacacagagagctccggggtgcatggctccaccctccaccgaaacatcctcagcagtgacctcattaaaatcatctatgtggatgggaacacccaggccctgctggacacagagctgcctggaggagacaaagctgatgcatccctgttggatccccgcgtgggcatccgctcggtgtgtgtcagccccaatggacagcatctagcatcaggggaccgtatgggcacacttagggtgcacgaacttcagtccctgagtgagatgctgaaggtggaggcccatgactctgagattctgtgcctggagtattctaagccagacacaggtctgaaactgctagcatcggcgagccgggaccggctgatccatgtgctggatgccgggcgggagtacagcctacagcagacgctggacgaacactcatcctccatcactgctgtcaagtttgcagccagtgatgggcaagtccgcatgatcagctgtggagcagacaagagcatctacttccgcactgcgcagaagtctggagatggagtgcagttcacacggacacaccacgtggtgcggaagacgaccctctatgacatggatgtggagcccagctggaagtacacggctatcggctgccaggaccgaaatattcggatatttaacatcagcagtggaaagcagaagaagctgtttaaagggtcacagggtgaggacggcacactcattaaggtgcagacagacccctcagggatctacattgccaccagctgttctgacaagaatctctccatttttgacttctcctcaggcgagtgcgtggccaccatgtttggccactcagagattgtcactggcatgaaatttagtaatgattgtaaacatctcatctctgtgtctggggacagctgcatatttgtgtggcgcctgagctctgagatgaccatcagcatgaggcagcgtctggccgagttgcgccagcgtcagcggggcggcaagcagcaaggaccatcctctccccaaagggcttctggacccaaccggcaccaggccccatcaatgctgtctcctggaccggctctctcatcagacagtgacaaggagggagaagatgaggggactgaagaagaacttccagcactgcccgtccttgccaagagtaccaagaaggcactggcctcggtccccagcccagctttgccccgaagcctgtcccactgggagatgagtcgggcacaggagtccgtggggttcctggacccagctcctgcagccaacccaggacccagaagaagagggcgctgggttcagccaggtgtggaactgagcgttagatccatgctggatctgcggcagctggaaacactggccccaagcctgcaggaccctagccaggactcgctggccatcatcccatctggtcccaggaagcatgggcaggaggcccttgagacttcactcactagccagaatgaaaagccccctcggcctcaggcttcccaaccttgttcctatccccatattatccgattattgtcacaagaggaaggggtctttgcccaagatctggaacctgcacccattgaagatggtattgtctacccggagccgagtgacaaccccaccatggataccagtgagttccaagtgcaggctccagcccggggaactctgggaagagtgtacccaggcagcaggagctcagaaaagcacagccctgacagtgcctgctctgtggattacagcagcagctgcctttccagcccggagcaccccactgaagactctgagagcacggagcccctcagtgtggatggcatctcctcagaccttgaagagccagctgagggtgatgaagaagaggaagaagaggagggaggcatgggcccctatgggctacaggagggcagcccccagactccagaccaggagcagtttctaaaacagcactttgagactctggccagtggagctgctccaggggccccagtgcaggtcccagagaggtcagagtctcggagtatctcttcacgattcctgttgcaagtacagacccgcccactcagggaaccatccccatcctcctcaagcctggcactgatgtcgagaccagcccaggtgccacaggcatctggtgagcagccgagaggcaatggtgccaatccccctggagcacccccggaggtggaaccgtcctctggcaaccccagcccccagcaggcagcctctgtgctgttgccacgatgccgtctcaaccctgacagcagctgggctcccaagagagtggccacagccagccccttttctggactccagaaggcccagtctgtgcacagtctggtgccacaggaaagacatgaggccagtctgcaggccccttcaccaggcgcactgctgtctcgggagatcgaagctcaggatggtctgggctccctgcccccagctgatggccctccgtctcggcctcactcctatcagaaccccaccaccagttccatggccaagatatcccgcagtatctctgttggggagaacctgggcctggtggctgaacctcaagctcatgcccccatccgagtctcaccactcagcaagctggccctgcccagccgggctcacctggtcctggacatccccaaaccactgcctgaccgtcctaccctggctgcattctctcctgtcaccaaaggccgggcccctggcgaggcagaaaagcctggcttcccggtgggcctaggaaaagctcacagtacaactgagagatgggcctgtttgggggagggcaccactcccaagcctaggacagagtgccaggctcatcctgggcccagcagcccctgtgcccagcaactgccagtcagcagcctcttccaaggccctgaaaacttgcagcccccaccccctgagaagactcccaaccccatggaatgcaccaagccaggggcagccctgagccaggactcagcggtgagcctggagcagtgtgagcagctggtggcagagctccgcggcagcgtgcgccaggcagtgcggctctaccactcggtggctggctgcaagatgccctcagcagagcaaagtcggattgcccagctcctcagagacaccttctcttcagtgcgacaggagctggaagctgtggctggggcagtgctgtccagcccaggcagcagccctggggctgtgggagccgagcagacacaggccctgctggagcaatactcagaactgttgcttcgagccgtggaacggcgtatggaacgcaaactctga

SEQ ID NO: 9 - DNA, 1881 bp, Homo sapiens
atggcagaccagagaatggacatttcttcaaccatcagtgatttcatgtccccgggccccaccgacctgctttccagctctcttggtaccagtggtgtggattgcaaccgcaaacggaaaggcagctccactgactaccaagaaagcatggacacagacaaagatgaccctcatggaaggttagaatatacagaacaccaaggaaggataaaaaatgcaagggaagctcacagtcagattgaaaagcggcgtcgggataaaatgaacagttttatagatgaattggcttctttggtaccaacatgcaacgcaatgtccaggaaattagataaacttactgtgctaaggatggctgttcagcacatgaaaacattaagaggtgccaccaatccatacacagaagcaaactacaaaccaacttttctatcagacgatgaattgaaacacctcattctcagggcagcagatggatttttgtttgtcgtaggatgtgaccgagggaagatactctttgtctcagagtctgtcttcaagatcctcaactacagccagaatgatctgattggtcagagtttgtttgactacctgcatcctaaagatattgccaaagtcaaggagcagctctcctcctctgacaccgcaccccgggagcggctcatagatgcaaaaactggacttccagttaaaacagatataacccctgggccatctcgattatgttctggagcacgacgttctttcttctgtaggatgaagtgtaacaggccttcagtaaaggttgaagacaaggacttcccctctacctgctcaaagaaaaaagcagatcgaaaaagcttctgcacaatccacagcacaggctatttgaaaagctggccacccacaaagatggggctggatgaagacaacgaaccagacaatgaggggtgtaacctcagctgcctcgtcgcaattggacgactgcattctcatgtagttccacaaccagtgaacggggaaatcagggtgaaatctatggaatatgtttctcggcacgcgatagatggaaagtttgtttttgtagaccagagggcaacagctattttggcatatttaccacaagaacttctaggcacatcgtgttatgaatattttcaccaagatgacataggacatcttgcagaatgtcataggcaagttttacagacgagagaaaaaattacaactaattgctataaatttaaaatcaaagatggttcttttatcacactacggagtcgatggttcagtttcatgaacccttggaccaaggaagtagaatatattgtctcaactaacactgttgttttagccaacgtcctggaaggcggggacccaaccttcccacagctcacagcatccccccacagcatggacagcatgctgccctctggagaaggtggcccaaagaggacccaccccactgttccagggattccagggggaacccgggctggggcaggaaaaataggccgaatgattgctgaggaaatcatggaaatccacaggataagagggtcatcgccttctagctgtggctccagcccattgaacatcacgagtacgcctccccctgatgcctcttctccaggaggcaagaagattttaaatggagggactccagacattccttccagtggcctactatcaggccaggctcaggagaacccaggttatccatattctgatagttcttctattcttggtgagaacccccacataggtatagacatgattgacaacgaccaaggatcaagtagtcccagtaatgatgaggcagcaatggctgtcatcatgagcctcttggaagcagatgctggactgggtggccctgttgactttagtgacttgccatggccgctgtaa

SEQ ID NO: 10 - DNA, 2181 bp, Homo sapiens
atggaagatagactccacatggataatggactggtaccccaaaagattgtgtcggtgcacttgcaggactccactctgaaggaagttaaggatcaggtctcaaacaagcaagcccagatcctagagccgaagcctgaaccttctcttgagattaagcctgagcaggacggtatggagcatgttggcagagatgacccaaaggctcttggtgaagaacccaaacaaaggagaggcagtgcctctgggagtgagcctgctggggacagtgacaggggagggggccccgttgagcattatcacctccatctgtctagttgccacgagtgtctggaacttgagaacagcaccattgagtcagtcaagtttgcgtctgccgagaacattccagaccttccctacgattatagcagcagtttggagagtgttgctgatgagacctcccccgaaagagaagggaggagagtcaacctcacgggaaaggcacccaacatcctcctctatgtgggctccgactcccaggaagccctcggccggttccacgaggtccggtctgtgctggccgactgtgtggacattgacagttatattctctaccacctgctggaggacagtgctctcagagacccgtggacggacaactgtctgctgttggtcattgctaccagggagtccattcccgaagacctgtaccagaagttcatggcctatctttctcagggagggaaggtgttgggcctgtcttcatccttcacctttggtggctttcaggtgacaagcaagggtgcactgcacaagacagtccagaacttggttttctccaaggctgaccagagcgaggtgaagctcagcgtcttgagcagtggctgcaggtaccaggaaggccccgtccggctcagccccggcaggctccagggccacctggagaatgaggacaaggacaggatgattgtgcatgtgccttttggaactcgcgggggagaagctgttctttgccaggtgcacttagaactacctcccagctccaacatagtgcaaactccagaagattttaacttgctcaagtcaagcaattttagaagatacgaagtccttagagagattctgacaacccttggcctcagctgtgacatgaaacaagttcctgccttaactcctctttacttgctgtcagctgcggaggaaatcagggatcctcttatgcagtggcttgggaaacatgtggactccgagggagaaataaaatccggccagctctctcttagatttgtttcatcctacgtgtctgaagtagaaataaccccatcttgtatacctgtggtgaccaacatggaggccttctcatcagaacatttcaacttagagatctatcgccaaaatctgcagaccaagcagttggggaaagtaattttgtttgccgaagtgacccccacaacgatgcgtctcctggatgggctgatgtttcagacaccgcaggaaatgggcttaatagtgatcgcggcccggcagaccgagggcaaaggacggggagggaatgtgtggctgagccctgtgggatgtgctctttctactctgctcatctccattccactgagatcccagctgggacagaggatcccgtttgtccagcatctgatgtccgtggctgtcgtggaagcagtgaggtccattcccgagtatcaggatatcaacttacgagtgaagtggcccaacgatatttattacagtgacctcatgaagatcggcggagttctggttaactcaacactcatgggagaaacattttatatacttattggctgtggatttaatgtgactaacagtaaccctaccatctgcatcaacgacctcatcacagaatacaataaacaacacaaggcagaactgaagcccttaagagccgattatctcatcgccagagtcgtgactgtgctggagaaactgatcaaagagtttcaggacaaagggcccaacagcgtccttcccctttattaccgatactgggtccacagtggtcagcaagtccatctgggcagcgcagagggaccaaaggtgtccatcgttggcctggacgattctggcttcctccaggttcaccaggagggcggcgaggttgtgactgtgcacccggacggcaactccttcgacatgctgagaaacctcatcctccccaaacggcggtaa

SEQ ID NO: 11 - protein, 6 aa, Homo sapiens
  MISC_FEATURE: Partial oligopeptide of JNK3 showing high score in the local alignment between JNK3 and KIAA1491 (SEQ ID NO6)
QGFDKQ

SEQ ID NO: 12 - protein, 6 aa, Homo sapiens
  MISC_FEATURE: Partial oligopeptide of KIAA1491 (SEQ ID NO6) showing high score in the local alignment between JNK3 and KIAA1491
QTFDKQ

SEQ ID NO: 13 - protein, 6 aa, Homo sapiens
  MISC_FEATURE: a peptide consisting of sequential 6 amino acid residues which exists in an amino acid sequence of JNK3
SLFPAD

SEQ ID NO: 14 - protein, 6 aa, Homo sapiens
  MISC_FEATURE: a partial peptide of KIAA0596(SEQ ID NO3), which shows high homology with the peptide of SEQ ID NO8
SLPPAD

SEQ ID NO: 15 - protein, 6 aa, Homo sapiens
  MISC_FEATURE: a peptide consisting of sequential 6 amino acid residues which exists in an amino acid sequence of JNK3
KVIEQL

SEQ ID NO: 16 - protein, 6 aa, Homo sapiens
  MISC_FEATURE: a partial peptide of BMAL1 or BMAL2, which shows high homology with the peptide of SEQ ID NO10
KVKEQL

SEQ ID NO: 17 - protein, 6 aa, Homo sapiens
  MISC_FEATURE: a peptide consisting of sequential 6 amino acid residues which exists in an amino acid sequence of JNK3
LPPSSS

SEQ ID NO: 18 - protein, 6 aa, Homo sapiens
  MISC_FEATURE: a peptide consisting of sequential 6 amino acid residues which exists in an amino acid sequence of JNK3
ANLCQV

SEQ ID NO: 19 - protein, 6 aa, Homo sapiens
  MISC_FEATURE: a partial peptide of BPL1, which shows high homology with the peptide of SEQ ID NO12
LPPSSN

SEQ ID NO: 20 - protein, 6 aa, Homo sapiens
  MISC_FEATURE: a partial peptide of BPL1, which shows high homology with the peptide of SEQ ID NO13
AVLCQV

SEQ ID NO: 21 - protein, 33 aa, Homo sapiens
  MISC_FEATURE: Partial oligopeptide of JNK3 showing high score in the local alignment between JNK3 and KIAA1491(SEQ ID NO6)
QPSPSGAAVNSSESLPPSSSVNDISSMSTDQTL

SEQ ID NO: 22 - protein, 33 aa, Homo sapiens
  MISC_FEATURE: Partial oligopeptide of KIAA1491(SEQ ID NO6) showing high score in the local alignment between JNK3 and KIAA1491
QSSATFSTAATSVSSSASSGVSLSSSMNTANSL

SEQ ID NO: 23 - protein, 43 aa, Homo sapiens
  MISC_FEATURE: Partial oligopeptide of JNK3 showing high score in the local alignment between JNK3 and KIAA1491(SEQ ID NO6)
SPSGAAVNSSESLPPSSSVNDISSMSTDQTLASDTDSSLEASA

SEQ ID NO: 24 - protein, 43 aa, Homo sapiens
  MISC_FEATURE: Partial oligopeptide of KIAA1491(SEQ ID NO6) showing high score in the local alignment between JNK3 and KIAA1491
AATSVSSSASSGVSLSSSMNTANSLCLGGTPASASSSSSRAAP

SEQ ID NO: 25 - protein, 4 aa, Homo sapiens
  MISC_FEATURE: Partial sequence identical in the sequences of JNK3 and KIAA1491(SEQ ID NO6)
FDKQ

SEQ ID NO: 26 - protein, 14 aa, Homo sapiens
  MISC_FEATURE: Partial oligopeptide of JNK3 showing high score in the local alignment between JNK3 and KIAA1491 (SEQ ID NO6)
HSAGIIHRDLKPSN

SEQ ID NO: 27 - protein, 14 aa, Homo sapiens
  MISC_FEATURE: Partial oligopeptide of KIAA1491(SEQ ID NO6) showing high score in the local alignment between JNK3 and KIAA1491
HPTTTTSWDLKPPT

SEQ ID NO: 28 - protein, 4 aa, Homo sapiens
  MISC_FEATURE: Partial sequence identical in the sequences of JNK3 and KIAA1491(SEQ ID NO6)
DLKP

SEQ ID NO: 29 - protein, 30 aa, Homo sapiens
  MISC_FEATURE: Partial oligopeptide of JNK3 showing high score in the local alignment between JNK3 and KIAA1491 (SEQ ID NO6)
PSGAAVNSSESLPPSSSVNDISSMSTDQTL

SEQ ID NO: 30 - protein, 29 aa, Homo sapiens
  MISC_FEATURE: Partial oligopeptide of KIAA1491(SEQ ID NO6) showing high score in the local alignment between JNK3 and KIAA1491
PYQSPVSSSESAPGTIMNGHGGGRSQQTL

SEQ ID NO: 31 - protein, 4 aa, Homo sapiens
  MISC_FEATURE: Partial sequence identical in the sequences of JNK3 and KIAA1491(SEQ ID NO6)
SSES

SEQ ID NO: 32 - protein, 33 aa, Homo sapiens
  MISC_FEATURE: Partial oligopeptide of JNK3 showing high score in the local alignment between JNK3 and KIAA1491 (SEQ ID NO6)
SGAAVNSSESLPPSSSVNDISSMSTDQTLASDT

SEQ ID NO: 33 - protein, 33 aa, Homo sapiens
  MISC_FEATURE: Partial oligopeptide of KIAA1491(SEQ ID NO6) showing high score in the local alignment between JNK3 and KIAA1491
TGDLTSSPLSQLSSSLSSHQSSLSAHAALSSST

SEQ ID NO: 34 - protein, 44 aa, Homo sapiens
  MISC_FEATURE: Partial oligopeptide of JNK3 showing high score in the local alignment between JNK3 and KIAA1491(SEQ ID NO6)
PSPSGAAVNSSESLPPSSSVNDISSMSTDQTLASDTDSSLEASA

SEQ ID NO: 35 - protein, 45 aa, Homo sapiens
  MISC_FEATURE: Partial oligopeptide of KIAA1491(SEQ ID NO6) showing high score in the local alignment between JNK3 and KIAA1491
SSPLSQLSSSLSSHQSSLSAHAALSSSTSHTHASVESASSHQSSA

SEQ ID NO: 36 - protein, 31 aa, Homo sapiens
  MISC_FEATURE: Partial oligopeptide of JNK3 showing high score in the local alignment between JNK3 and KIAA0596(SEQ ID NO3)
QPSPSGAAVNSSESLPPSSSVNDISSMSTDQ

SEQ ID NO: 37 - protein, 31 aa, Homo sapiens
  MISC_FEATURE: Partial oligopeptide of  KIAA0596(SEQ ID NO3) showing high score in the local alignment between JNK3 and KIAA0596
QPPPPEKTPNPMECTKPGAALSQDSAVSLEQ

SEQ ID NO: 38 - protein, 9 aa, Homo sapiens
  MISC_FEATURE: Partial oligopeptide of JNK3 showing high score in the local alignment between JNK3 and KIAA0596 (SEQ ID NO3)
FTPQKTLEE

SEQ ID NO: 39 - protein, 9 aa, Homo sapiens
  MISC_FEATURE: Partial oligopeptide of  KIAA0596(SEQ ID NO3) showing high score in the local alignment between JNK3 and KIAA0596
YSLQQTLDE

SEQ ID NO: 40 - protein, 20 aa, Homo sapiens
  MISC_FEATURE: Partial oligopeptide of JNK3 showing high score in the local alignment between JNK3 and KIAA0596(SEQ ID NO3)
SDCTLKILDFGLARTAGTSF

SEQ ID NO: 41 - protein, 20 aa, Homo sapiens
  MISC_FEATURE: Partial oligopeptide of  KIAA0596(SEQ ID NO3) showing high score in the local alignment between JNK3 and KIAA0596
SDKNLSIFDFSSGECVATMF

SEQ ID NO: 42 - protein, 15 aa, Homo sapiens
  MISC_FEATURE: Partial oligopeptide of JNK3 showing high score in the local alignment between JNK3 and KIAA0596(SEQ ID NO3)
KLKASQARDLLSKML

SEQ ID NO: 43 - protein, 15 aa, Homo sapiens
  MISC_FEATURE: Partial oligopeptide of  KIAA0596(SEQ ID NO3) showing high score in the local alignment between JNK3 and KIAA0596
KLLASASRDRLIHVL

SEQ ID NO: 44 - protein, 23 aa, Homo sapiens
  MISC_FEATURE: Partial oligopeptide of JNK3 showing high score in the local alignment between JNK3 and KIAA0596(SEQ ID NO3)
SLFPADSEHNKLKASQARDLLSK

SEQ ID NO: 45 - protein, 23 aa, Homo sapiens
  MISC_FEATURE: Partial oligopeptide of  KIAA0596(SEQ ID NO3) showing high score in the local alignment between JNK3 and KIAA0596
SLVPQERHEASLQAPSPGALLSR

SEQ ID NO: 46 - protein, 17 aa, Homo sapiens
  MISC_FEATURE: Partial oligopeptide of JNK3 showing high score in the local alignment between JNK3 and KIAA0596 (SEQ ID NO3)
IEEWKELIYKEVMNSEE

SEQ ID NO: 47 - protein, 18 aa, Homo sapiens
  MISC_FEATURE: Partial oligopeptide of  KIAA0596(SEQ ID NO3) showing high score in the local alignment between JNK3 and KIAA0596
LDKWVELRVYPEVKDSNQ

SEQ ID NO: 48 - protein, 14 aa, Homo sapiens
  MISC_FEATURE: Partial oligopeptide of JNK3 showing high score in the local alignment between JNK3 and KIAA0596(SEQ ID NO3)
SSMSTDQTLASDTD

SEQ ID NO: 49 - protein, 14 aa, Homo sapiens
  MISC_FEATURE: Partial oligopeptide of  KIAA0596(SEQ ID NO3) showing high score in the local alignment between JNK3 and KIAA0596
SMLSPGPALSSDSD

SEQ ID NO: 50 - protein, 23 aa, homo sapiens
  misc_feature: Partial oligopeptide of JNK3 showing high score in the local alignment between JNK3 and BMAL1
SKSKVDNQFYSVEVGDSTFTVLK

SEQ ID NO: 51 - protein, 23 aa, homo sapiens
  misc_feature: Partial oligopeptide of BMAL1 showing high score in the local alignment between JNK3 and BMAL1
TREKITTNCYKFKIKDGSFITLR

SEQ ID NO: 52 - protein, 14 aa, homo sapiens
  misc_feature: Partial oligopeptide of JNK3 showing high score in the local alignment between JNK3 and BMAL1
VGDSTFTVLKRYQN

SEQ ID NO: 53 - protein, 14 aa, homo sapiens
  misc_feature: Partial oligopeptide of BMAL1 showing high score in the local alignment between JNK3 and BMAL1
VSESVFKILNYSQN

SEQ ID NO: 54 - protein, 10 aa, homo sapiens
  misc_feature: Partial oligopeptide of JNK3 showing high score in the local alignment between JNK3 and BMAL1
EQLGTPCPEF

SEQ ID NO: 55 - protein, 10 aa, homo sapiens
  misc_feature: Partial oligopeptide of BMAL1 showing high score in the local alignment between JNK3 and BMAL1
ELLGTSCYEY

SEQ ID NO: 56 - protein, 22 aa, homo sapiens
  misc_feature: Partial oligopeptide of JNK3 showing high score in the local alignment between JNK3 and BMAL1
SSMSTDQTLASDTDSSLEASAG

SEQ ID NO: 57 - protein, 22 aa, homo sapiens
  misc_feature: Partial oligopeptide of BMAL1 showing high score in the local alignment between JNK3 and BMAL1
SSPSNDEAAMAVIMSLLEADAG

SEQ ID NO: 58 - protein, 10 aa, homo sapiens
  misc_feature: Partial oligopeptide of JNK3 showing high score in the local alignment between JNK3 and BMAL1
SDCTLKILDF

SEQ ID NO: 59 - protein, 10 aa, homo sapiens
  misc_feature: Partial oligopeptide of BMAL1 showing high score in the local alignment between JNK3 and BMAL1
SESVFKILNY

SEQ ID NO: 60 - protein, 10 aa, homo sapiens
  misc_feature: Partial oligopeptide of JNK3 showing high score in the local alignment between JNK3 and BMAL1
YIDQWNKVIE

SEQ ID NO: 61 - protein, 10 aa, homo sapiens
  misc_feature: Partial oligopeptide of BMAL1 showing high score in the local alignment between JNK3 and BMAL1
FMNPWTKEVE

SEQ ID NO: 62 - protein, 11 aa, homo sapiens
  misc_feature: Partial oligopeptide of JNK3 showing high score in the local alignment between JNK3 and BMAL1
VKGQPSPSGAA

SEQ ID NO: 63 - protein, 11 aa, homo sapiens
  misc_feature: Partial oligopeptide of BMAL1 showing high score in the local alignment between JNK3 and BMAL1
VKEQLSSSDTA

SEQ ID NO: 64 - protein, 31 aa, homo sapiens
  misc_feature: Partial oligopeptide of JNK3 showing high score in the local alignment between JNK3 and BMAL2
EEKTKNGVVKGQPSPSGAAVNSSESLPPSSS

SEQ ID NO: 65 - protein, 31 aa, homo sapiens
  misc_feature: Partial oligopeptide of BMAL2 showing high score in the local alignment between JNK3 and BMAL2
DDSSPTGLMKDTHTVNCRSMSNKELFPPSPS

SEQ ID NO: 66 - protein, 10 aa, homo sapiens
  misc_feature: Partial oligopeptide of JNK3 showing high score in the local alignment between JNK3 and BMAL2
EQLGTPCPEF

SEQ ID NO: 67 - protein, 10 aa, homo sapiens
  misc_feature: Partial oligopeptide of BMAL2 showing high score in the local alignment between JNK3 and BMAL2
ELLGTSCYEY

SEQ ID NO: 68 - protein, 23 aa, homo sapiens
  misc_feature: Partial oligopeptide of JNK3 showing high score in the local alignment between JNK3 and BMAL2
SKSKVDNQFYSVEVGDSTFTVLK

SEQ ID NO: 69 - protein, 23 aa, homo sapiens
  misc_feature: Partial oligopeptide of BMAL2 showing high score in the local alignment between JNK3 and BMAL2
SKEKILTDSYKFRAKDGSFVTLK

SEQ ID NO: 70 - protein, 12 aa, homo sapiens
  misc_feature: Partial oligopeptide of JNK3 showing high score in the local alignment between JNK3 and BMAL2
SKSKVDNQFYSV

SEQ ID NO: 71 - protein, 12 aa, homo sapiens
  misc_feature: Partial oligopeptide of BMAL2 showing high score in the local alignment between JNK3 and BMAL2
SKKKEHRKFYTI

SEQ ID NO: 72 - protein, 8 aa, homo sapiens
  MISC_FEATURE: Partial oligopeptide of JNK3 showing high score in the local alignment between JNK3 and BPL1
SYLLYQML

SEQ ID NO: 73 - protein, 8 aa, homo sapiens
  MISC_FEATURE: Partial oligopeptide of BPL1 showing high score in the local alignment between JNK3 and BPL1
SYILYHLL

SEQ ID NO: 74 - protein, 8 aa, homo sapiens
  MISC_FEATURE: Partial oligopeptide of JNK3 showing high score in the local alignment between JNK3 and BPL1
SEPTLDVK

SEQ ID NO: 75 - protein, 8 aa, homo sapiens
  MISC_FEATURE: Partial oligopeptide of BPL1 showing high score in the local alignment between JNK3 and BPL1
PEPSLEIK

SEQ ID NO: 76 - protein, 14 aa, homo sapiens
  MISC_FEATURE: Partial oligopeptide of JNK3 showing high score in the local alignment between JNK3 and BPL1
IHRDLKPSNIVVKS

SEQ ID NO: 77 - protein, 14 aa, homo sapiens
  MISC_FEATURE: Partial oligopeptide of BPL1 showing high score in the local alignment between JNK3 and BPL1
VHLELPPSSNIVQT

SEQ ID NO: 78 - protein, 9 aa, homo sapiens
  MISC_FEATURE: Partial oligopeptide of JNK3 showing high score in the local alignment between JNK3 and BPL1
QKTLEEFQD

SEQ ID NO: 79 - protein, 9 aa, homo sapiens
  MISC_FEATURE: Partial oligopeptide of BPL1 showing high score in the local alignment between JNK3 and BPL1
EKLIKEFQD

SEQ ID NO: 80 - protein, 4 aa, homo sapiens
  MISC_FEATURE: Partial sequence identical in the sequences of JNK3 and BPL1
EFQD

SEQ ID NO: 81 - protein, 12 aa, homo sapiens
  MISC_FEATURE: Partial oligopeptide of JNK3 showing high score in the local alignment between JNK3 and BPL1
DANLCQVIQMEL

SEQ ID NO: 82 - protein, 11 aa, homo sapiens
  MISC_FEATURE: Partial oligopeptide of BPL1 showing high score in the local alignment between JNK3 and BPL1
EAVLCQVHLEL

SEQ ID NO: 83 - protein, 4 aa, homo sapiens
  MISC_FEATURE: Partial sequence identical in the sequences of JNK3 and BPL1
LCQV

SEQ ID NO: 84 - protein, 7 aa, homo sapiens
  MISC_FEATURE: Partial oligopeptide of JNK3 showing high score in the local alignment between JNK3 and BPL1
LPPSSSV

SEQ ID NO: 85 - protein, 7 aa, homo sapiens
  MISC_FEATURE: Partial oligopeptide of BPL1 showing high score in the local alignment between JNK3 and BPL1
LPPSSNI

SEQ ID NO: 86 - protein, 5 aa, homo sapiens
  MISC_FEATURE: Partial sequence identical in the sequences of JNK3 and BPL1
LPPSS

SEQ ID NO: 87 - protein, 15 aa, homo sapiens
  MISC_FEATURE: Partial oligopeptide of JNK3 showing high score in the local alignment between JNK3 and BPL1
PQKTLEEFQDVYLVM

SEQ ID NO: 88 - protein, 15 aa, homo sapiens
  MISC_FEATURE: Partial oligopeptide of BPL1 showing high score in the local alignment between JNK3 and BPL1
SQEALGRFHEVRSVL

SEQ ID NO: 89 - protein, 11 aa, homo sapiens
  MISC_FEATURE: Partial oligopeptide of JNK3 showing high score in the local alignment between JNK3 and BPL1
KTLEEFQDVYL

SEQ ID NO: 90 - protein, 11 aa, homo sapiens
  MISC_FEATURE: Partial oligopeptide of BPL1 showing high score in the local alignment between JNK3 and BPL1
RSIPEYQDINL